(19) United States
(12) Patent Application Publication
(10) Pub. No.: US 2025/0319281 A1
(43) Pub. Date: Oct. 16, 2025

(54) TRANSCATHETER THERAPY IMAGE GUIDANCE USING TRACKERS

(71) Applicant: GE Precision Healthcare LLC, Waukesha, WI (US)

(72) Inventors: Eigil Samset, Horten (NO); Régis Vaillant, Villebon sur Yvette (FR); Olivier Gérard, Oslo (NO)

(21) Appl. No.: 18/633,375

(22) Filed: Apr. 11, 2024

(51) Int. Cl. A61M 25/01; A61B 6/03; A61B 8/00

(52) U.S. Cl. CPC A61M 25/0105; A61B 6/03; A61B 8/4254; A61B 8/465; A61M 2025/0166

(57) ABSTRACT

Methods and systems are disclosed to perform automated multiplanar reformation (MPR) on an image volume generated by an imaging system, based on position and orientation data received from sensors positioned in interventional tools or devices used in the procedure, such as a probe and/or a delivery catheter. A desired plane for performing the MPR may be automatically configured based on the sensors. The sensors may be advantageously used to automatically select a view plane that includes the catheter tip and/or target anatomies of the patient and/or maintain the catheter tip and/or target anatomies in view more rapidly and efficiently than may be accomplished using manual MPR.

### FIELD

The present description relates generally to interventional image guidance, and more particularly, to methods and systems for optimal visualization and navigation of an interventional tool to a target region during interventional procedures.

### BACKGROUND

Medical ultrasound is an imaging modality that employs ultrasound waves propagating through the internal structures of a body of a patient to produce a corresponding image. For example, an ultrasound probe comprising a plurality of transducer elements emits ultrasonic waves which reflect or echo, refract, or are absorbed by structures in the body. Medical ultrasound modalities, such as echocardiograms, are used prior to, during, and/or after procedures to image internal structures relevant to the procedure. For example, for certain cardiac procedures such as transcatheter therapies that employ the use of a catheter and/or other devices, ultrasound images such as echocardiogram images may be used to guide positioning of the catheter. Other supplementary imaging systems such as a fluoroscopy imaging system, a computed tomography (CT) imaging system, and/or a magnetic resonance (MR) imaging system may additionally be used to aid guidance of the catheter.

### BRIEF DESCRIPTION

In one embodiment, a method for an interventional imaging system comprises, during an image-guided interventional procedure performed by an operator of the interventional imaging system on a patient, receiving a first image from a probe of an ultrasound system, the first image oriented with respect to a first coordinate system of the probe; receiving a second image from a supplementary imaging system of the interventional imaging system, the second image oriented with respect to a second coordinate system different from the first coordinate system; receiving a first set of position and orientation data from a first sensor located in the probe, the first set of position and orientation data acquired with respect to a third coordinate system of the first sensor, the third coordinate system different from the first coordinate system and the second coordinate system; reorienting at least one of the first image and the second image to a common coordinate system shared by both of the first image and the second image, based on the first set of position and orientation data; and displaying the first image and the second image within the common coordinate system on a display device.

It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1A schematically shows a schematic diagram of an interventional imaging system, in accordance with one or more embodiments of the present disclosure;

FIG. 1B schematically shows a pictorial view of the interventional imaging system including a fluoroscopy imaging system and an ultrasound imaging system, in accordance with one or more embodiments of the present disclosure;

FIG. 2 shows a field of view intra-cardiac ultrasound imaging probe during a cardiac transcatheter therapy task, in accordance with one or more embodiments of the present disclosure;

FIG. 3 shows a first schematic data flow diagram showing a flow of data through the interventional imaging system when generating images to display on a display device, in accordance with one or more embodiments of the present disclosure;

FIG. 4 shows an image processing system of the interventional imaging system, in accordance with one or more embodiments of the present disclosure;

FIG. 5 shows a second schematic data flow diagram showing a flow of data through the interventional imaging system when generating images to display on a display device, in accordance with one or more embodiments of the present disclosure;

FIG. 6 is a flowchart illustrating a method for displaying images generated by the interventional imaging system in a common coordinate system, in accordance with one or more embodiments of the present disclosure;

FIG. 7 shows a first exemplary set of images displayed on a display device of the interventional imaging system during a cardiac transcatheter therapy task, where the set of images are oriented based on different coordinate systems, in accordance with one or more embodiments of the present disclosure;

FIG. 8 shows a second exemplary set of images displayed on the display device of the interventional imaging system during the cardiac transcatheter therapy task, where the set of images are oriented in accordance with a common coordinate system, in accordance with one or more embodiments of the present disclosure;

FIG. 9 shows an exemplary menu of templates for performing MPR on an image volume, in accordance with one or more embodiments of the present disclosure; and

FIG. 10 shows a fifth exemplary set of images displayed on a display device of the interventional imaging system, where an ultrasound image of a delivery catheter is replaced by a CAD model of the delivery catheter, in accordance with one or more embodiments of the present disclosure.

### DETAILED DESCRIPTION

Interventional techniques are widely used for managing a plurality of life-threatening medical conditions. Particularly, certain interventional techniques entail minimally invasive image-guided procedures that provide a cost-effective alternative to invasive surgery. Additionally, the minimally invasive interventional procedures minimize pain and trauma caused to a patient, thereby resulting in shorter hospital stays. Accordingly, minimally invasive transcatheter therapies have found extensive use, for example, in treatment of valvular and congenital heart diseases. The transcatheter therapies may be further facilitated through multi-modality imaging that aids in planning, guidance, and evaluation of procedure related outcomes and complications.

As an example, interventional procedures such as transesophageal echocardiography (TEE) and/or intracardiac echocardiography (ICE) may be used to provide high resolution images of intracardiac anatomy. The high resolution images, in turn, allow for real-time guidance of interventional devices during structural heart disease (SHD) interventions such as transcatheter aortic valve implantation (TAVI), paravalvular regurgitation repair, and/or mitral valve interventions.

TEE may be used to diagnose and/or treat SHD and/or electrophysiological disorders such as arrhythmias. To that end, TEE employs a probe positioned inside the esophagus of a patient to visualize cardiac structures. Although TEE allows for well-defined workflows and good image quality, TEE may not be suitable for all cardiac interventions. For example, TEE may provide limited visualization of certain anterior cardiac features due to imaging artifacts caused due to shadowing from surrounding structures and/or a lack of far-field exposure. Accordingly, in some interventional procedures, ICE may be used to provide higher resolution images of cardiac structures, often under conscious sedation of the patient.

Typically, during an ICE-assisted interventional procedure, an ICE catheter may be inserted into a vein, such as the femoral vein, navigated to a right atrium of a heart of the patient, in some cases using a trans-septal puncture technique to advance into a left side of the heart to image a cardiac region of interest (ROI). The ICE catheter may include a transducer array configured to generate volumetric images of the cardiac ROI corresponding to the interventional procedure being performed. The ICE images, thus generated, may be used to provide a medical practitioner with real-time guidance for positioning and/or navigating an interventional device such as a clip, a valve, a closure device, an ablation catheter, or a needle within the patient's body. For example, the ICE images may be used to provide the medical practitioner with an illustrative map to navigate the ablation catheter within the patient's body to provide therapy to desired regions of interest (ROIs). Additionally, the images may be used, for example, to obtain basic cardiac measurements, visualize valve structure, and measure septal defect dimensions to aid the medical practitioner in accurately diagnosing a medical condition of the patient.

The ICE equipment may be interfaced with other interventional imaging systems, thus allowing for supplemental imaging that may provide additional information for device guidance, diagnosis, and/or treatment. For example, a CT imaging system may be used to provide supplemental views of an anatomy of interest based on pre-operative images to facilitate ICE-assisted interventional procedures. That is, a first set of images (2D ultrasound images and/or 3D renderings of an ultrasound image volume) of the cardiac structures may be generated from an ICE catheter and displayed on a display device during operation of the ICE, while a second set of one or more images of the cardiac structures may be provided by a CT imaging system.

The supplemental imaging may also include other types of views or images. For example, a fluoroscopy image may be additionally or alternatively displayed on the display device concurrently with either or both of the first and second sets of images. The fluoroscopy image may aid an operator of the ICE catheter in determining a location and orientation of the ICE catheter with respect to the ROI. The fluoroscopy image is a 2D projection that may be updated in real-time during the procedure. However, the use of fluoroscopy may be reduced as much as possible to reduce an amount of radiation dose during the procedure. The amount of radiation dose may be proportional to an amount of time taken by the procedure, whereby it may be desirable to reduce the amount of time. Additional benefits of reducing the amount of time is that a number of patients that are treated in a day may be increased, and a per-patient cost of performing interventional procedures may be reduced.

One factor influencing the amount of time taken by the procedure is that maneuvering and/or orienting the ICE catheter to acquire a desired view of a cardiac ROI relevant to a current patient exam, and maneuvering a catheter delivery system for a device using images for guidance may be difficult. Acquiring the desired view of the cardiac ROI may include determining an optimal view of one or more anatomical structures of interest for performing the interventional procedure. Accordingly, in conventional ICE (or TEE) systems, the medical practitioner may manually configure one or more controls of the ICE system to adjust a view direction of one or more images to display a clinically useful view. However, this may be a complicated and time consuming procedure involving trial and error. Furthermore, manual configuration of the system controls may interrupt the interventional procedure and prolong a duration of the procedure. The prolonged procedure time may increase a risk of trauma to the cardiac tissues.

One reason that the manual configuration of the system controls may result in a complicated and time consuming procedure is that different views of the cardiac ROI displayed on the display device may have different orientations. In other words, a first view of the cardiac structures generated from the ICE catheter or TEE probe may be displayed with a first orientation based on a first coordinate system of the ICE catheter or TEE probe; a second view generated by the CT imaging system may be displayed with a second orientation based on a second coordinate system, such as a first patient coordinate system established by a table of the CT imaging system; and a third view of a fluoroscopy image may be displayed with a third orientation based on a third coordinate system, such as a second patient coordinate system established by a table of the fluoroscopy imaging system; where the first, second, and third coordinate systems may be different. As a result, it may be difficult for the operator to harmonize the three views into a mental image of where the ICE catheter is with respect to the cardiac ROI, and the operator may rely on a cumbersome manual identification of anatomical landmarks for registration.

To address this problem, and to reduce the amount of time taken by such procedures, systems and methods are proposed herein to reorient one or more views such that the first, second, third, and/or additional views displayed on the display device are shown with respect to a same coordinate system. By showing the first, second, third, and/or additional views in the same coordinate system, it may be easier for operators to visualize a relative positioning of elements used in the procedure (the catheter, probe, guide wires, device, etc.) with respect the cardiac ROI, thereby reducing the amount of time taken by the operators to perform the procedure.

Current approaches to harmonizing coordinate systems of different views typically rely on registering the coordinate systems based on an identified orientation of the catheter (or probe) in a fluoroscopy image. However, a solution that reduces a reliance on fluoroscopy would be preferable to limit radiation as well as to simplify running multiple imaging systems simultaneously. Additionally, planar reformations prescribed by the echocardiography may be suboptimal in crisply delineated interventional tools, such as catheters, due to a blooming effect, where the tip may appear blurry or with an exaggerated size. The interventional tools may also be out-of-plane.

An alternative approach is disclosed herein, where to reorient the one or more views such that the first, second, third, and/or additional views displayed on the display device are shown with respect to a same coordinate system, sensors may be advantageously positioned in one or more of the elements used in the procedure. Moving elements of the interventional system can be tracked by integrating sensors inside them. The sensors may be tracked to determine the precise 3D positions and orientation of the elements used in the procedure, from which coordinate systems of the different views and images may be harmonized, in accordance with methods described herein. By calibrating and registering the co-ordinate systems of the sensors with the co-ordinate system of the imaging systems (which can be done during manufacturing and/or during installation of the interventional system), all relevant data (image volumes as well as catheters) can be expressed in the same co-ordinate system. The fluoroscopic images are projection images without depth resolution (depth along the x-ray beam). The orientation and (center) location of the fluoroscopic images are intrinsically tracked by the fluoroscopy imaging system 151, and can be expressed in the sensors co-ordinate system using the abovementioned calibration process.

Further, the sensors may be used to partially or totally automate performing multiplanar reformation (MPR), where image data of a 3D volume is reformatted into one or more 2D planes to be displayed on the display device to visualize the cardiac ROI. The 3D image volume may be a CT image volume, an ultrasound image volume, an MR image volume, or an image volume of a different supplementary imaging system. MPR is typically a manual procedure, where the operator adjusts controls of the imaging system to select a desired plane of the 3D image volume to be visualized. For example, the operator may select a display of the 3D image volume that includes standard reference planes (e.g., coronal, axial, and sagittal). The operator may manually select a point in one of the standard reference planes, for example, at an anatomical landmark of the cardiac ROI. The operator may manually rotate the 3D image volume about an axis defined by the point to display oblique views of 3D image volume. The operator may iteratively select points in and/or adjust/rotate each of the oblique views until the desired view/plane is achieved, in a trial and error fashion.

By including a sensor in the catheter and in the ICE or TEE probe, a position and orientation of a tracked device may be automatically determined, and one or more desired planes may be automatically configured based on the sensors. For example, a first plane of the 3D image volume may be defined by the position and orientation of the catheter, such that the first plane is perpendicular to a forward direction of the catheter. The first plane may be displayed on the display device. A second plane of the 3D image volume may be defined in parallel to the orientation of the catheter, with the catheter tip included in the second plane. The second plane may be displayed on the display device. A third plane of the 3D image volume may be defined based on a combination of the position and orientation of the catheter, and one or more anatomical landmarks, where the anatomical landmarks may be manually selected or automatically selected. A third plane may be displayed on the display device. A fourth plane of the 3D image volume may be defined based the orientation of the C-arm so that the plan is parallel or orthogonal to a direction of a projected fluoroscopic image. Further, the views generated by the first, second, third and fourth planes may be displayed concurrently on the display device. Other combinations of the position and orientation of the catheter, probe, C-arm and anatomical landmarks may be devised to define the planes to be displayed on the display device. In this way, the sensors may be advantageously used to automatically select a view plane that includes the catheter tip and/or target anatomies of the patient more rapidly and efficiently than may be accomplished using manual MPR.

Further, other sensors may be included in other tools or elements used during a procedure. For example, a procedure may include using a probe and a delivery catheter, where the probe is used to visualize the delivery catheter as the catheter delivers a device, such as a valve or clip, to a target location. A first sensor may be included in the probe; a second sensor may be included in a tip of the delivery catheter; and a third sensor may be included in the device. Each of the first sensor, the second sensor, and the third sensor may share a same coordinate system, based on a shared field generator used by the sensors. During the procedure, one or more of a first position of the first sensor, a second position of a second sensor, and a third position of the third sensor may be used to generate a fourth plane. Thus, a combination of various sensor positions, the orientation of the catheter and/or probe, and one or more anatomical landmarks may be advantageously used to generate a coordinated set of views that may be registered to a same coordinate system.

In this way, by including sensors in the catheter, the device, and/or other elements of the procedure, the MPR may be performed automatically or semi-automatically, with less user input from the operator. The views generated using the sensors may be displayed in a same coordinate system, where a motion of the camera, catheter, device, cardiac structures, or other elements of the views may be aligned. Additionally, a precision of an orientation of the views may be greater than if the MPR were performed manually.

It should be appreciated that although embodiments of the present disclosure are described with reference to ICE, use of the present systems and methods in other imaging applications and/or modalities is also contemplated. For example, the present systems and methods may be implemented in Transthoracic echocardiography (TTE) systems, TEE systems, intravascular ultrasound (IVUS) systems, and/or Optical Coherence Tomography (OCT) systems. Embodiments of the present systems and methods may also be used to more accurately diagnose and stage coronary artery disease and to help monitor therapies including, high intensity focused ultrasound (HIFU), radiofrequency ablation (RFA), catheter ablation, and brachytherapy by providing an optimal view of the target structure that allows for more accurate structural and functional measurements.

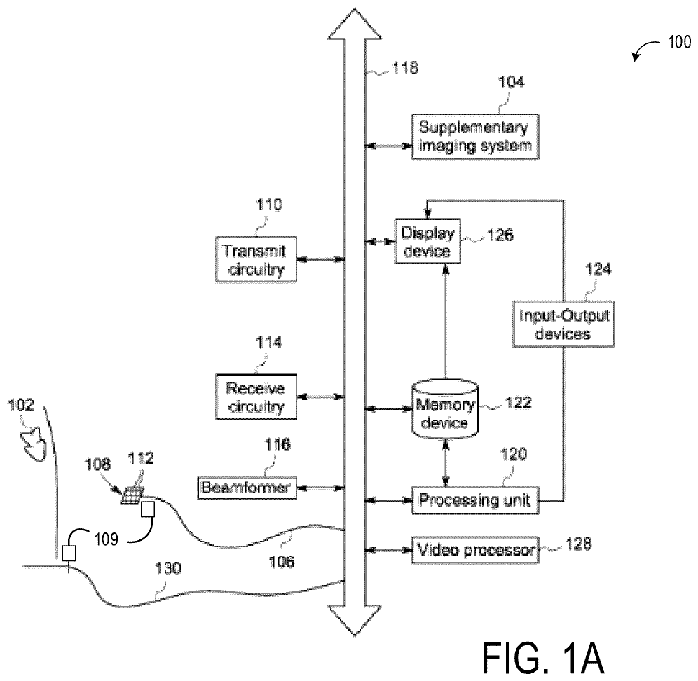

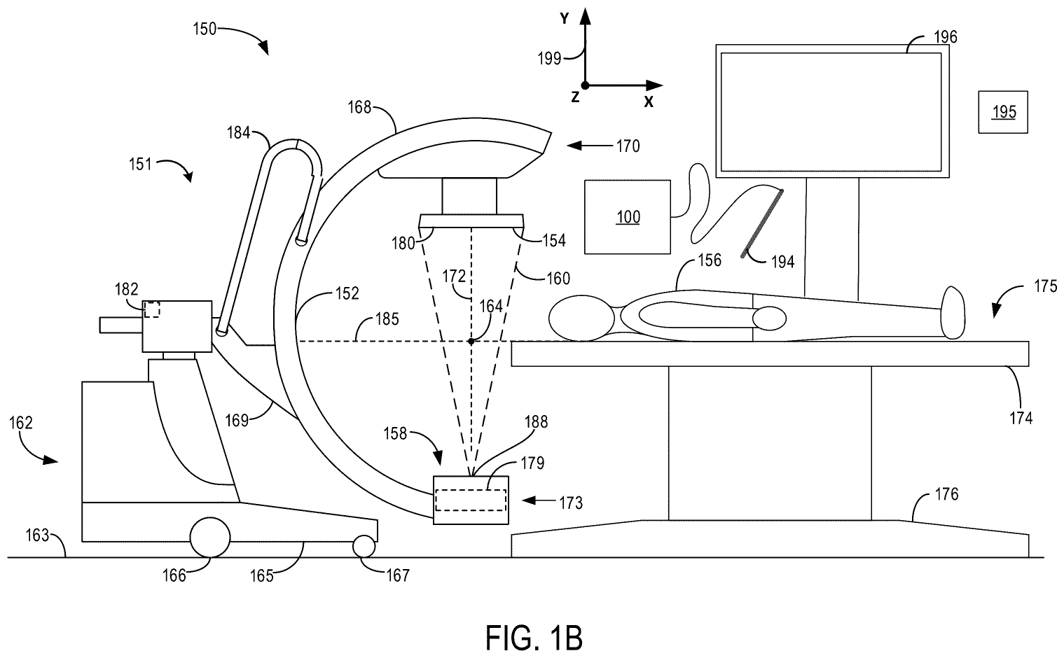

Referring now to the figures, FIG. 1A illustrates an exemplary imaging system 100 for visualization of a target structure 102 for use during interventional procedures. For discussion purposes, the system 100 is described with reference to an ICE system. However, as previously noted, in certain embodiments, the system 100 may be implemented in other interventional imaging systems such as a TTE system, a TEE system, an OCT system, a magnetic resonance imaging (MRI) system, a CT system, a positron emission tomography (PET) system, and/or an X-ray system. Additionally, it may be noted that although the present embodiment is described with reference to imaging a cardiac region corresponding to a patient, certain embodiments of the system 100 may be used with other biological tissues such as lymph vessels, cerebral vessels, and/or in non-biological materials.

In one embodiment, the system 100 employs ultrasound signals to acquire image data corresponding to the target structure 102 in a subject. Moreover, the system 100 may combine the acquired image data corresponding to the target structure 102, for example the cardiac region, with supplementary image data. The supplementary image data, for example, may include previously acquired images (e.g., pre-operative images) and/or real-time intra-operative image data generated by a supplementary imaging system 104 such as a CT, MRI, PET, ultrasound, fluoroscopy, electrophysiology, and/or X-ray system. In some examples, the supplementary imaging system may be a second ultrasound imaging system (e.g., an ICE may be used along with a TEE, etc.). Specifically, a combination of the acquired image data, and/or supplementary image data may allow for generation of a composite image that provides a greater volume of medical information for use in accurate guidance for an interventional procedure and/or for providing more accurate anatomical measurements.

Accordingly, in one embodiment, the system 100 includes an imaging device such as an endoscope, a laparoscope, a needle, a probe, and/or a catheter 106. The catheter 106 is adapted for use in a confined medical or surgical environment such as a body cavity, orifice, or chamber corresponding to a subject. The catheter 106 may further include at least one imaging subsystem 108 disposed at a distal end of the catheter 106. The imaging subsystem 108 may be configured to generate cross-sectional images of the target structure 102 for evaluating one or more corresponding characteristics. Particularly, in one embodiment, imaging subsystem 108 is configured to acquire a series of three-dimensional (3D) ultrasound images corresponding to the subject. In certain embodiments, the system 100 may be configured to generate the 3D model relative to time, thereby generating a 4D model or volume corresponding to the target structure such as the heart of the patient. The system 100 may use the 3D and/or 4D image data, for example, to visualize a 4D model of the target structure 102 for providing a medical practitioner with real-time guidance for navigating a delivery catheter within one or more chambers of the heart.

To that end, in certain embodiments, the imaging subsystem 108 includes transmit circuitry 110 that may be configured to generate a pulsed waveform to drive an array of transducer elements 112. Particularly, the pulsed waveform drives the array of transducer elements 112 to emit ultrasonic pulses into a body or volume of interest in the subject. At least a portion of the ultrasonic pulses generated by the transducer elements 112 back-scatter from the target structure 102 to produce echoes that return to the transducer elements 112 and are received by receive circuitry 114 for further processing.

In one embodiment, the receive circuitry 114 may be operatively coupled to a beamformer 116 that may be configured to process the received echoes and output corresponding radio frequency (RF) signals. Although FIG. 1A illustrates the transducer elements 112, the transmit circuitry 110, the receive circuitry 114, and the beamformer 116 as distinct elements, in certain embodiments, one or more of these elements may be implemented together as an independent acquisition subsystem in the system 100. The acquisition subsystem may be configured to acquire image data corresponding to the subject, such as a patient, for further processing. As used herein, subject refers to any human or animal subject that may be imaged using the present system.

Further, the system 100 includes a processing unit 120 communicatively coupled to the acquisition subsystem over a communications network 118. The processing unit 120 may be configured to receive and process the acquired image data, for example, the RF signals according to a plurality of selectable ultrasound imaging modes in near real-time and/or offline mode. To that end, the processing unit 120 may be operatively coupled to the beamformer 116, the transducer probe 116, and/or the receive circuitry 114. In one example, the processing unit 120 may include devices such as one or more general-purpose or application-specific processors, digital signal processors, microcomputers, microcontrollers, Application Specific Integrated Circuits (ASICs), Field Programmable Gate Arrays (FPGA), or other suitable devices in communication with other components of the system 100.

In certain embodiments, the processing unit 120 may be configured to provide control and timing signals for selectively configuring one or more imaging and/or viewing parameters for performing a desired imaging task. By way of example, the processing unit 120 may be configured to automatically adjust FOV, spatial resolution, frame rate, depth, and/or frequency of ultrasound signals used for imaging the target structure 102.

Moreover, in one embodiment, the processing unit 120 may be configured to store the acquired volumetric images, the imaging parameters, and/or viewing parameters in a memory device 122. The memory device 122, for example, may include storage devices such as a random access memory, a read only memory, a disc drive, solid-state memory device, and/or a flash memory. Additionally, the processing unit 120 may display the volumetric images and or information derived from the image to a user, such as a cardiologist, for further assessment.

Accordingly, in certain embodiments, the processing unit 120 may be coupled to one or more input-output devices 124 for communicating information and/or receiving commands and inputs from the user. The input-output devices 124, for example, may include devices such as a keyboard, a touchscreen, a microphone, a mouse, a control panel, a display device 126, a foot switch, a hand switch, and/or a button. In one embodiment, the display device 126 may include a graphical user interface (GUI) for providing the user with configurable options for imaging desired regions of the subject. By way of example, the configurable options may include a selectable volumetric image, a selectable ROI, a desired scan plane, one or multiple selectable 2D cross sections (or multi planar reconstructions) of the volumetric image, a delay profile, a designated pulse sequence, a desired pulse repetition frequency, and/or other suitable system settings used to image the desired ROI. Additionally, the configurable options may include a choice of image-derived information to be communicated to the user. The image-derived information, for example, may include a position and/or orientation of an interventional device, a magnitude of strain, and/or a determined value of stiffness in a target region estimated from the received signals.

In one embodiment, the processing unit 120 may be configured to process the RF signal data to generate the requested image-derived information based on user input. Particularly, the processing unit 120 may be configured to process the RF signal data to generate 2D, 3D, and/or four-dimensional (4D) datasets based on specific scanning and/or user-defined requirements. Additionally, in certain embodiments, the processing unit 120 may be configured to process the RF signal data to generate the volumetric images in real-time while scanning the target region and receiving corresponding echo signals. As used herein, the term “real-time” may be used to refer to an imaging rate upwards of about 10 volumetric images per second with a delay of less than 1 second. Additionally, in one embodiment, the processing unit 120 may be configured to customize the delay in reconstructing and rendering the volumetric images based on specific system-based and/or application-specific requirements. Further, the processing unit 120 may be configured to process the RF signal data such that a resulting image is rendered, for example, at the rate of 30 volumetric images per second on the associated display device 126 that is communicatively coupled to the processing unit 120.

In one embodiment, the display device 126 may be a local device. Alternatively, the display device 126 may be remotely located to allow a remotely located medical practitioner to track the image-derived information corresponding to the subject. In certain embodiments, the processing unit 120 may be configured to update the volumetric images on the display device 126 in an offline and/or delayed update mode. Particularly, the volumetric images may be updated in the offline mode based on the echoes received over a determined period of time. Alternatively, the processing unit 120 may be configured to dynamically update the volumetric images and sequentially display the updated volumetric images on the display device 126 as and when additional volumes of ultrasound data are acquired.

With continued reference to FIG. 1A, in certain embodiments, the system 100 may further include a video processor 128 that may be configured to perform one or more functions of the processing unit 120. For example, the video processor 128 may be configured to digitize the received echoes and output a resulting digital video stream on the display device 126. In one embodiment, the video processor 128 may be configured to display the volumetric images on the display device 126, for example, using a Cartesian coordinate system. Particularly, as described in more detail below, one or more of the system 100, the supplementary imaging system 104, and/or the catheter 106 may be calibrated and/or registered to a common coordinate system to allow for visualization of a change in a view of the target structure 102 with a corresponding change in the position and/orientation of the catheter 106. Accordingly, the display device 126 may be used to provide real-time feedback to the medical practitioner regarding a current view corresponding to the target structure 102 and/or an interventional device 130 (e.g., a delivery catheter, such as an ablation catheter) employed to perform intervention at a site corresponding to the target structure 102.

However, visualizing the structures within the chambers of the heart in a desired view determined to be suitable for a patient exam being undertaken may be a challenging procedure. A high degree of freedom corresponding to the imaging subsystem 108 disposed at the distal end of the catheter 106 may complicate maneuvering and/or orienting the ICE catheter 106 within open cavities of the heart. Optimally positioning the imaging subsystem 108 to acquire image data corresponding to the desired FOV of the target structure 102, therefore, may be complicated and may often depend upon a skill and experience of a cardiologist. Even an experienced cardiologist, however, may expend a substantial amount of time to manually configure system controls to acquire a clinically acceptable view of the target structure 102. The substantial time taken to manually configure the system controls may interrupt the interventional procedure, while impeding real-time diagnosis and/or guidance of the interventional device 130.

Embodiments of the present system 100, however, allow for automatic processing of acquired volumetric images to visualize the target structure 102 in the desired view without employing repeated manual reconfigurations of the system controls. The desired view may correspond to an imaging plane that satisfies one or more statutory, clinical, application-specific, and/or user-defined specifications, thereby allowing for real-time tracking of the interventional device 130, accurate measurements of the patient anatomy, and/or efficient evaluation of the target structure 102.

Specifically, the video processor 128 may be configured to process the acquired volumetric image to automatically reposition and/or reorient the volumetric image to adjust a visualization of the target structure 102. In particular, the video processor 128 may be configured to process the acquired volumetric image to automatically reposition and/or reorient the volumetric image based on an output of one or more sensors 109 included in catheter 106 (for example, at imaging subsystem 108), and/or in interventional device 130. For example, the output of the one or more sensors 109 may be used to adjust the volumetric image from a first coordinate system to a second, different coordinate system. The output of the one or more sensors 109 may be used to automatically or semi-automatically perform MPR on the volumetric image to adjust the visualization of the target structure 102. The output of the one or more sensors 109 may also be used to visualize a catheter as a 3D object within a 3D rendering of an image volume of a target anatomy.

The video processor 128 may also be configured to supplement the optimal view of the target structure 102 with additional views of the target structure 102 that are acquired by the supplementary imaging system 104. As previously noted, use of the additional views may aid in providing more definitive information corresponding to the target structure 102. Accordingly, in one embodiment, the video processor 128 may be configured to display a composite volumetric image that combines the reoriented and/or repositioned view of the anatomical structures with the supplementary views.

FIG. 1B shows an exemplary interventional imaging system 150 that includes the ultrasound imaging system 100 of FIG. 1A, and a fluoroscopy imaging system 151 as a supplementary imaging system 104 used during interventional imaging, such as during a transcatheter therapy task. The fluoroscopy imaging system 151 includes a C-arm 152 (e.g., a C-shaped gantry), an X-ray unit or tube 158 positioned opposite to an X-ray detector 154 and configured to emit X-ray radiation. In other examples, the radiation source may be configured to emit a different type of radiation for imaging (e.g., imaging a subject, such as patient 156), such as gamma rays, and the X-ray detector 154 may be configured to detect the radiation emitted by the radiation source (e.g., X-ray beam 160). The fluoroscopy imaging system 151 additionally includes base unit 162 supporting fluoroscopy imaging system 151 on ground surface 163 on which the fluoroscopy imaging system 151 sits (e.g., via base 165 supported by wheel 166, wheel 167, etc.).

The C-arm 152 may include a C-shaped portion 168 connected to an extended portion 169, with the extended portion 169 rotatably coupled to the base unit 162. The X-ray detector 154 is coupled to the C-shaped portion 168 at a first end 170 of the C-shaped portion 168, and the X-ray unit 158 is coupled to the C-shaped portion 168 at an opposing, second end 173 of the C-shaped portion 168. As an example, the C-arm 152 may be configured to rotate in opposing directions relative to the base unit 162. The C-arm 152 may be rotatable about at least a rotational axis 164 and may additionally rotate about axis 185. The C-shaped portion 168 may be rotated as described above in order to adjust the X-ray unit 158 and X-ray detector 154 (positioned on opposite ends of the C-shaped portion of the C-arm 152 along axis 172, where axis 172 intersects rotational axis 164 and extends radially relative to rotational axis 164) through a plurality of positions.

During an imaging operation (e.g., a scan), a portion of a patient's body placed in an opening formed between the X-ray unit 158 and X-ray detector 154 may be irradiated with radiation from the X-ray unit 158. For example, patient 156 may be supported by a patient support table 174, with the patient support table 174 including a support surface 175 and base 176, and may be arranged between the X-ray unit 158 and the X-ray detector 154. The X-ray unit 158 includes an X-ray tube insert 179 and X-ray radiation generated by the X-ray tube insert 179 may emit from the X-ray unit 158. The radiation may penetrate the portion of the patient's body arranged to be irradiated and may travel to the X-ray detector 154 where the radiation is captured (e.g., intercepted by a detector surface 180 of the X-ray detector 154). By penetrating the portion of the patient's body placed between the X-ray unit 158 and X-ray detector 154, an image of the patient's body is captured and relayed to an electronic controller 182 of the fluoroscopy imaging system 151 (e.g., via an electrical connection line, such as electrically conductive cable 184). The image may be displayed via a display device 196. Images of the subject acquired by the fluoroscopy imaging system 151 via the X-ray unit 158 and the X-ray detector 154 as described above may be referred to herein as projection images and/or scan projection images.

The base unit 162 may include the electronic controller (e.g., a control and computing unit) that processes instructions or commands sent from the user input devices during operation of the fluoroscopy imaging system 151. The base unit 162 may also include an internal power source (not shown) that provides electrical power to operate the fluoroscopy imaging system 151. Alternatively, the base unit 162 may be connected to an external electrical power source to power the fluoroscopy imaging system 151. A plurality of connection lines may be provided to transmit electrical power, instructions, and/or data between the X-ray unit 158, X-ray detector 154, and the control and computing unit. The plurality of connection lines may transmit electrical power from the electrical power source (e.g., internal and/or external source) to the X-ray unit 158 and X-ray detector 154.

The C-arm 152 may be adjusted to a plurality of different positions by a rotation, extension, or adjustment of the C-shaped portion 168 of the C-arm 152. For example, in an initial, first position shown by FIG. 1A, the X-ray detector 154 may be positioned vertically above the X-ray unit 158 relative to a ground surface 163 on which the fluoroscopy imaging system 151 sits, with axis 172 arranged normal to the ground surface 163 intersecting a midpoint of each of the outlet 188 of X-ray unit 158 and detector surface 113 of X-ray detector 154. The C-arm 152 may be adjusted from the first position to a different, second position by rotating the C-shaped portion 168. In one example, the X-ray unit 158 is positioned vertically above the rotational axis 164 of the C-shaped portion 168 of the C-arm 152, and the X-ray detector 154 may be positioned vertically below the rotational axis 164. Different rotational positions of the C-arm 152 are possible.

As described above in reference to FIG. 1A, during an image-guided, interventional imaging therapy task, a probe/catheter 194 of the ultrasound imaging system 100 may be inserted into the patient 156 while the patient 156 is positioned within the C-arm 152 and being imaged by the fluoroscopy imaging system 151. Thus, images generated by each of the ultrasound imaging system 100 and the fluoroscopy imaging system 151 may be displayed on the display device 196. The images may be displayed adjacent to each other, such that operators of the ultrasound imaging system 100 and the fluoroscopy imaging system 151 may view the images during performance of the therapy task. Additionally, other images may be displayed on the display device 196 that may aid the operators in performing the interventional imaging therapy task, such as CT images, MR images, or a different type of image, which may be generated from the patient 156 prior to performing the interventional imaging therapy task.

However, an orientation of the images displayed on the display device 196 may be different for different images. That is, one or more of the images (e.g., a fluoroscopy image) may be displayed with an orientation based on a patient coordinate system of the fluoroscopy imaging system 151, indicated by the reference coordinate axes 199. One or more of the other images (e.g., an ultrasound image, CT image, MR image, etc.) may not be displayed with an orientation based on the patient coordinate system, and may be displayed in one or more different coordinate systems. For example, the ultrasound image may be displayed with an orientation based on a coordinate system of an ultrasound probe of the ultrasound imaging system 100; the CT image may be displayed with an orientation based on a coordinate system of a CT imaging system used to generate the CT image; the MR image may be displayed with an orientation based on a coordinate system of an MR imaging system used to generate the MR image; and so on. As described below, the various images displayed on display device 196 may be harmonized to a shared coordinate system using one or more sensors included in probes, interventional tools, or devices of interventional imaging system 150, such as the probe 194. The one or more sensors may share a coordinate system based on a shared field generator 195.

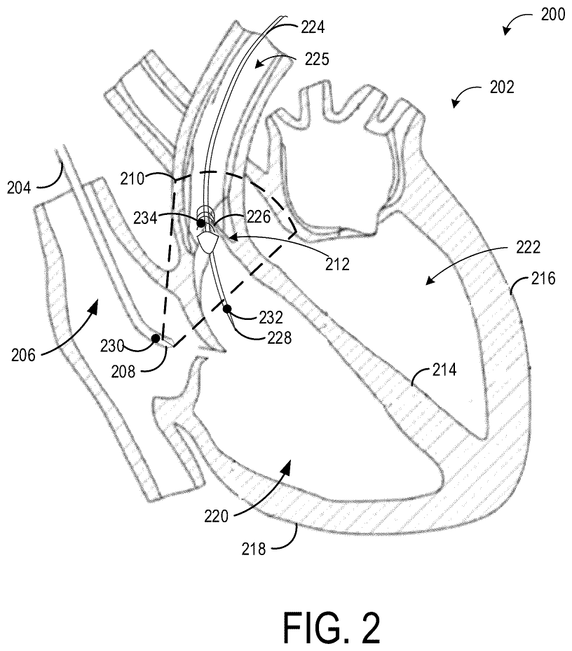

FIG. 2 shows an illustration 200 of a human heart 202 depicting a field of view of an ICE probe 204 of an imaging system (e.g., imaging system 100 of FIG. 1A) within a simplified cross section of the human heart 202. The ICE probe 204 is positioned in a right atrium 206 of the human heart 202, during a valve replacement procedure. The ICE probe 204 may be a non-limiting example of probe/catheter 106 of FIG. 1A, and may include an ultrasound transducer array 208 (e.g., array of transducer elements 112), which can image at least a portion of the heart 202. For example, an image viewing angle 210 afforded by the transducer array 208 may allow imaging a pulmonary valve 212, a septum 214, ventricular walls 216 and 218, a right ventricle 220, a left ventricle 222, and other structures. Insertion of the ICE probe 204 into a circulatory system vessel or other anatomical cavity via percutaneous cannulation is well known in the medical arts.

Additionally, a delivery catheter 224 has been inserted into a lumen 225 of a left pulmonary artery of the human heart 202 to place a replacement valve 226 within pulmonary valve 212. The delivery catheter may be a sheath catheter, a guide catheter, an ablation catheter, etc. A tip 228 of the delivery catheter 224 is depicted as having passed through the valve 212 into the right ventricle 220.

As mentioned above, the images on the display device 196 of FIG. 1B may be displayed oriented with respect to different coordinate systems, which may make it harder for an operator of the ultrasound imaging system to perform the surgical task. To address this problem, each of the ICE probe 204, the delivery catheter 224, and the replacement valve 226 may include a position and orientation tracking sensor (e.g., where the sensor measures the position and orientation with 6 degrees of freedom). That is, the ICE probe 204 may include a first sensor 230, which may track a first position (X, Y, and Z dimensions) and orientation (roll, pitch, and yaw measurements) of the ultrasound transducer array 208; the delivery catheter 224 may include a second sensor 232, which may track a position and orientation of the tip 228 of the delivery catheter 224; and the replacement valve 226 may include a third sensor 234, which may track a position and orientation of the replacement valve 226. By including sensors 230, 232, and 234 in the ICE probe 204, the delivery catheter 224, and the replacement valve 226, respectively, precise locations of the ICE probe 204, the delivery catheter 224, and the replacement valve 226 may be transmitted to the imaging system and used to control a display of images generated by the ultrasound transducer array 208 of ICE probe 204, and/or supplementary images of the heart 202 generated by one or more supplementary imaging systems (e.g., supplementary imaging system 104). As described in greater detail below, the precise locations may be advantageously used during an MPR process, to adjust a 2D view of a volumetric image of the human heart 202 used by the operator to guide the delivery catheter 224. The sensors 230, 232, and 234 may include electric position sensors, or fiber optic sensors, or a different type of sensor.

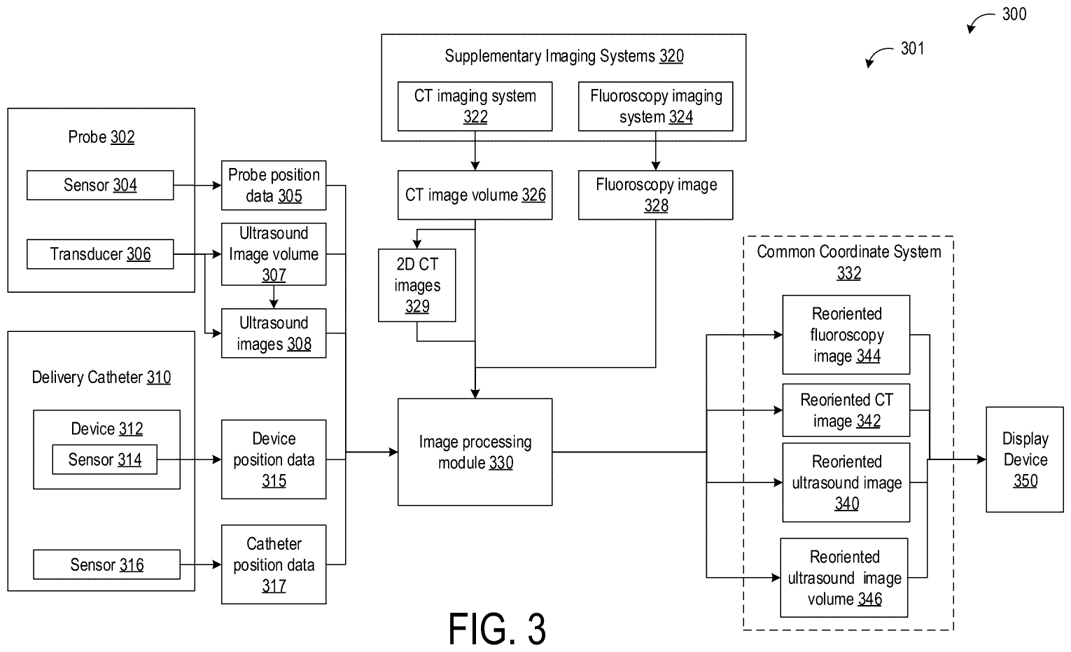

FIG. 3 shows an exemplary simplified data flow diagram 300 that depicts a flow of data within an imaging system 301 as images of a patient anatomy are generated on a display device 350 (e.g., display 196 of FIG. 1B) during an image-guided transcatheter therapy task. Imaging system 301 may be a non-limiting example of imaging system 100 of FIG. 1A. The images generated on the display device 350 may include ultrasound images generated by a transducer array 306 of a probe 302. The transducer array 306 (e.g., ultrasound transducer arrays 208 and 112 of FIGS. 1 and 2, respectively) may generate 2D or 3D ultrasound images. Probe 302 may be the same as or similar to probe/catheter 106 of FIG. 1A, and may comprise an endoscope, a laparoscope, a needle, or a different apparatus configured with an ultrasound transducer array.

The images generated on the display device 350 may include supplementary images from one or more supplementary imaging systems 320. For example, the one or more supplementary imaging systems 320 may include a CT imaging system 322 (e.g., fluoroscopy imaging system 151), which may generate a CT image volume 326 of a target anatomy of the image-guided transcatheter therapy task. The one or more supplementary imaging systems 320 may also include a fluoroscopy imaging system 324 (e.g., fluoroscopy imaging system 151), which may generate fluoroscopy images 328 of the target anatomy of the image-guided transcatheter therapy task. In other embodiments, supplementary imaging systems 320 may include additional or different imaging systems.

While the image-guided transcatheter therapy task is being performed, an operator of the imaging system 301 may insert the probe 302 within a body of a patient. As the probe 302 is manipulated within the body of the patient, ultrasound images 308 generated by the transducer array may be displayed on display device 350. The ultrasound images 308 may be 2D ultrasound images acquired when the probe 302 is a 2D ultrasound probe of the imaging system 301, or 2D views of a 3D ultrasound image volume 307 acquired when the probe 302 is a 3D ultrasound probe the imaging system 301.

The operator may navigate the probe 302 to the target anatomy based on one or more of the fluoroscopy images 328. The operator may be aided in navigating the probe 302 by one or more ultrasound images 308 generated by the probe, which may be displayed on the display device 350 concurrently with the fluoroscopy image 328. The operator may be further aided in navigating the probe 302 by a 2D CT image 329 of the CT image volume 326, which may be displayed on the display device 350 concurrently with the ultrasound images 308 and the fluoroscopy image 328. In some cases, the operator may be further aided by an atlas (not shown in FIG. 3), which could be a static x-ray image or a pre-operative CT image. As one example, a pre-operative CT scan may be performed with segmented vessel and segment structures, and a resulting CT atlas image may be co-registered to the fluoroscopy image 328. The CT atlas image could be shown side-by-side, or overlaid on one or more of the fluoroscopy image 328, the ultrasound image 308, or a different image displayed on the display device 350, where the tracked probe is shown on or in the CT atlas image.

As the operator navigates the probe 302, the operator may view the ultrasound image 308, the ultrasound image volume 307, the fluoroscopy image 328, and the 2D CT image 329 displayed concurrently on the display device 350. As the operator adjusts a position of the probe 302, the ultrasound image 308 and/or the ultrasound image volume 307 may be updated, based on the adjusted position. For example, as the operator adjusts the position of the probe 302, a desired plane of view of the operator may be redefined by a new orientation of the probe 302 with respect to elements of the target anatomy. An ultrasound image 308 corresponding to the desired plane of view may be displayed on the display device 350. The ultrasound image 308, 2D CT image 329, and fluoroscopy image 328 may be displayed in an adjacent configuration, such that the operator may view each of ultrasound image 308, 2D CT image 329, and fluoroscopy image 328 in proximity with each other at the same time.

During the image-guided transcatheter therapy task, the operator may insert a device 312 into the body of the patient using a delivery catheter 310. For example, the device may be a valve replacement, and the operator may insert the valve replacement into a valve of a heart of the patient, as described above in reference to FIG. 2. The operator may insert a device 312 into the body of the patient via a different route than the probe 302. For example, the probe 302 may be inserted through the esophagus or through the inferior vena cava of the patient, and the delivery catheter 310 may be inserted through the inferior vena cava or another vessel of the patient. As the operator inserts the device 312 into the body of the patient, the operator may adjust the position and/or orientation of the probe 302 to be able to see the delivery catheter 310.

However, navigating the delivery catheter 310 to the desired location and depositing the device 312 at the desired location using a combination of ultrasound image 308, ultrasound image volume 307, 2D CT image 329, and fluoroscopy image 328 for guidance may be complicated by the fact that one or more of the ultrasound image 308, ultrasound image volume 307, 2D CT image 329, and fluoroscopy image 328 may be oriented with respect to a different coordinate system. In other words, the ultrasound image 308 or the ultrasound image volume 307 may be generated by transducer array in accordance with a first coordinate system; the CT image 329 may be generated by CT imaging system 322 in accordance with a second coordinate system; and fluoroscopy image 328 may be generated by fluoroscopy imaging system 324 in accordance with a third coordinate system, where any of the first, second, and third coordinate systems may be different from each other. As a result, the operator who is relying on these images to navigate the delivery catheter 310 to the desired location may have to perform a number of mental rotations to understand how manipulating the delivery catheter 310 will affect a trajectory of the delivery catheter 310 and an ability to advance the delivery catheter 310 to the desired location. This may involve various attempts in a trial-and-error manner, and may prolong a duration of the procedure. Additionally, the misalignment of the first, second, and third coordinate systems may also result in miscommunication between the interventional cardiologist (e.g., the operator) and echocardiographer (also referred to as an imager), as they may be using language/terms that correspond to a reference coordinate system of one image, which may be incorrect for a different reference coordinate system.

An additional issue is that fluoroscopy and ultrasound images may be complementary, where fluoroscopy images may show dense structures with higher resolution than ultrasound images, while ultrasound images may show soft structures with higher resolution than fluoroscopy images. As a result, showing fluoroscopy images and ultrasound images in a common coordinate system may enable a fused display (e.g., an overlay), which may be helpful during curtain steps of the procedure. Such a fused display may not be possible when the fluoroscopy and ultrasound images are displayed in different coordinate systems.

To address the orientations of the different images used by the operator to guide the delivery catheter 310 with respect to different coordinate systems, one or more of the ultrasound image 308, the ultrasound image volume 307, the 2D CT image 329, and the fluoroscopy image 328 may be automatically reoriented by the imaging system 301 in accordance with a common coordinate system 332, to generate a set of reoriented views of the ultrasound image 308, the 2D CT image 329, and the fluoroscopy image 328. That is, ultrasound image 308 may be reoriented to generate reoriented ultrasound image 340; 2D CT image 329 may be reoriented to generate a reoriented 2D CT image 342; fluoroscopy image 328 may be reoriented to generate a reoriented fluoroscopy image 344; and/or the ultrasound image volume 307 may be reoriented to generate reoriented ultrasound image 346. One or more of the reoriented images 340, 342, 344, and 346 may be displayed on the display device 350 in accordance with the common coordinate system 332. Specifically, the ultrasound image 308, the 2D CT image 329, the fluoroscopy image 328, and the ultrasound image volume 307 may be replaced by the reoriented ultrasound image 340, the reoriented 2D CT image 342, the reoriented fluoroscopy image 344, and the reoriented image volume 346, respectively. When the reoriented images 340, 342, 344, and 346 are displayed on the display device in accordance with the common coordinate system 332, changes to the reoriented images 340, 342 and 344 due to an adjustment of the probe 302 and/or a movement of delivery catheter 310 may be synchronized.

It should be appreciated that in practice, not all of images 340, 342, 344, and 346 may be reoriented to a different, common coordinate system. Rather, the common coordinate system 332 may be one of the first coordinate system, the second coordinate system, and the third coordinate system, and one or more images corresponding to a coordinate system that is not the common coordinate system 332 may be reoriented to the common coordinate system 332.

The automatic reorientation of the one or more of the ultrasound image 308, the ultrasound image volume 307, the 2D CT image 329, and the fluoroscopy image 328 may be performed by an image processing module 330 of the imaging system 301. Image processing module 330 may be a non-limiting example of, or included within processing unit 120 of FIG. 1A. To perform the automatic reorientations, the image processing module 330 may determine absolute locations (e.g., with respect to a coordinate system) of the transducer array of the probe 302, the delivery catheter 310, and the device 312 within the body of the patient.

The location of the transducer array may be determined based on probe position data 305, which may be generated by a sensor 304 positioned within the probe 302 (e.g., sensor 230 of FIG. 2). Sensor 304 may be positioned within the probe 302 at a location of the transducer array, for example. The probe position data 305 may include a location of the transducer array in three dimensions (e.g., X, Y, and Z values), in accordance with a first coordinate system. The probe position data 305 may also include an orientation of the transducer array in three directions (e.g., pitch, roll, and yaw) in accordance with the first coordinate system.

Similarly, the location of the delivery catheter 310 may be determined based on catheter position data 317, which may be generated by a sensor 316 positioned within the delivery catheter 310 (e.g., sensor 232 of FIG. 2). In various embodiments, sensor 316 may be positioned at a tip (e.g., tip 228) of the delivery catheter 310. The catheter position data 317 may include a location of the delivery catheter 310 in three dimensions (e.g., X, Y, and Z values), in accordance with a second coordinate system. The second coordinate system may be the same as the first coordinate system, or different from the first coordinate system. The catheter position data 317 may also include an orientation of the delivery catheter 310 in three dimensions (e.g., pitch, roll, and yaw) in accordance with the second coordinate system.

The location of the device 312 may be determined based on device position data 315, which may be generated by a sensor 314 positioned at the device 312 (e.g., sensor 234 of FIG. 2). The device position data 315 may include a location of the device 312 in three dimensions (e.g., X, Y, and Z values), in accordance with a third coordinate system. The third coordinate system may be the same as the first and/or second coordinate systems, or different from the first and/or second coordinate systems. The device position data 315 may also include an orientation of the device 312 in three dimensions (e.g., pitch, roll, and yaw) in accordance with the third coordinate system.

The image processing module 330 may receive the probe position data 305, the device position data 315, and the catheter position data 317 from the sensors 304, 314, and 316, respectively. The one or more of the probe position data 305, the device position data 315, and the catheter position data 317 may be transmitted wirelessly to the image processing module 330 from the sensors 304, 314, and 316, respectively, or via an electrical wire, or via fiber optic cable, or in a different manner. The image processing module 330 may also receive the ultrasound image volume 307, the 2D CT image 329 and/or the fluoroscopy image 328 corresponding to a location of the probe 302 at a time when the probe position data 305 is received. The 2D CT image 329 may be oriented in accordance with a fourth coordinate system of the CT imaging system 322, and the fluoroscopy image 328 may be oriented with respect to a fifth coordinate system of the fluoroscopy imaging system 324, where either or both of the fourth and fifth coordinate systems may be different from any or all of the first, second, and third coordinate systems of the probe 302, the delivery catheter 310, and the device 312.

The image processing module may reorient one or more of the ultrasound image 308, the ultrasound image volume 307, the 2D CT image 329, and the fluoroscopy image 328 such that the reoriented ultrasound image 340, the reoriented CT image 342, the reoriented fluoroscopy image 344, and the reoriented ultrasound image volume 346 may all be displayed in accordance with the common coordinate system 332.

For example, the fluoroscopy image 328 may be acquired according to a patient coordinate system defined by an orientation of a table of the fluoroscopy imaging system 324 with respect to a C-arm of the fluoroscopy imaging system 324 (e.g., table 174 and C-arm 152 of FIG. 1B, respectively). The ultrasound image 308 may be acquired according to a coordinate system defined by an orientation of the probe 302. Prior to displaying the fluoroscopy image 328 and the ultrasound image 308 on the display device 350, the patient coordinate system may be defined as the common coordinate system 332. The ultrasound image 308 may be reoriented such that the reoriented ultrasound image 340 is displayed on the display device 350 in accordance with the common (e.g., patient) coordinate system. The 2D CT image 329 may be similarly reoriented, and may be displayed also in the patient coordinate system. As a result of displaying the reoriented ultrasound image 340 and the 2D CT image 329 in the same, common coordinate system as the fluoroscopy imaging system 324, it may be more intuitively understandable for the operator to manipulate the catheter and to understand how the anatomic, devices and other structures all relate spatially to each other. This may lead to a greater understanding of where the delivery catheter 310 is within the body of the patient, and where an anatomical target is and how to get there, without having to do mental rotations in a cumbersome and time-consuming trial-and-error fashion.

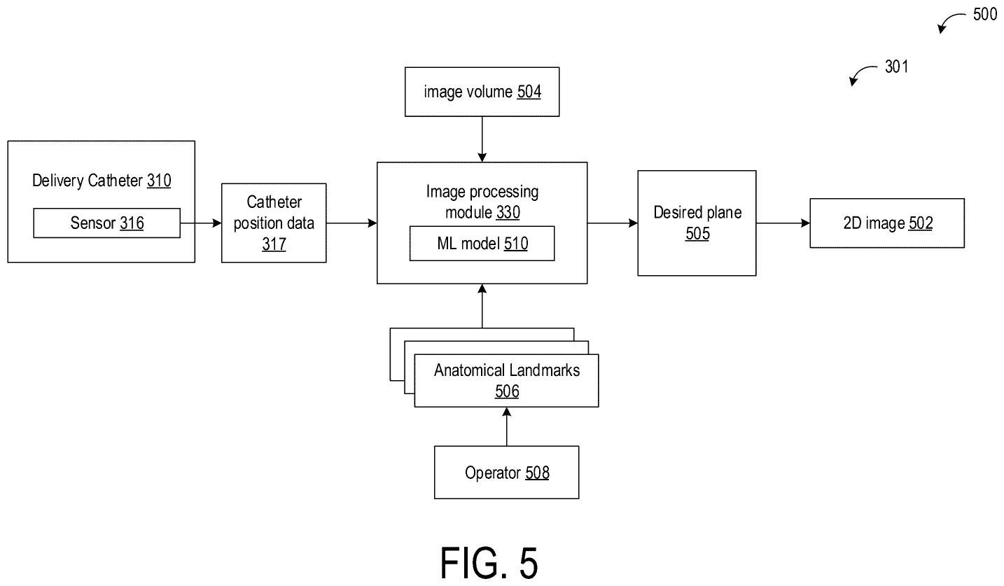

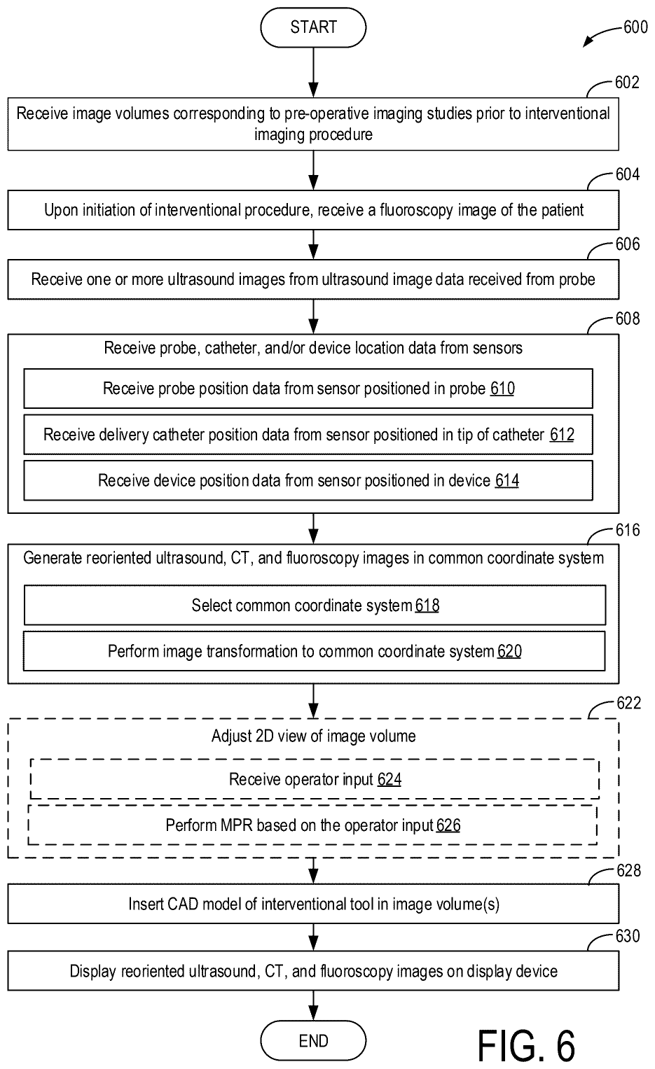

In various examples, the ultrasound image 308, the 2D CT image 329, and the fluoroscopy image 328 may be reoriented by following one or more steps of the method described in reference to FIG. 6 below. In addition, as shown in FIG. 5, sensors 304, 314, and 316 may be advantageously used to perform MPR to view 3D image volumes, such as the ultrasound image volume 307 or the CT image volume 326.

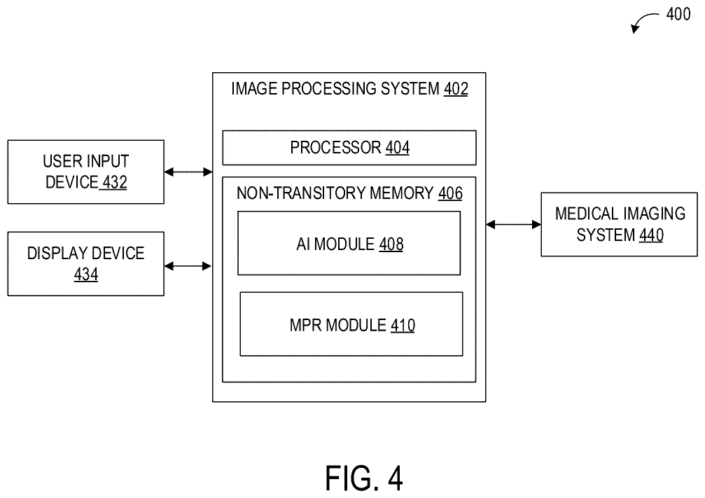

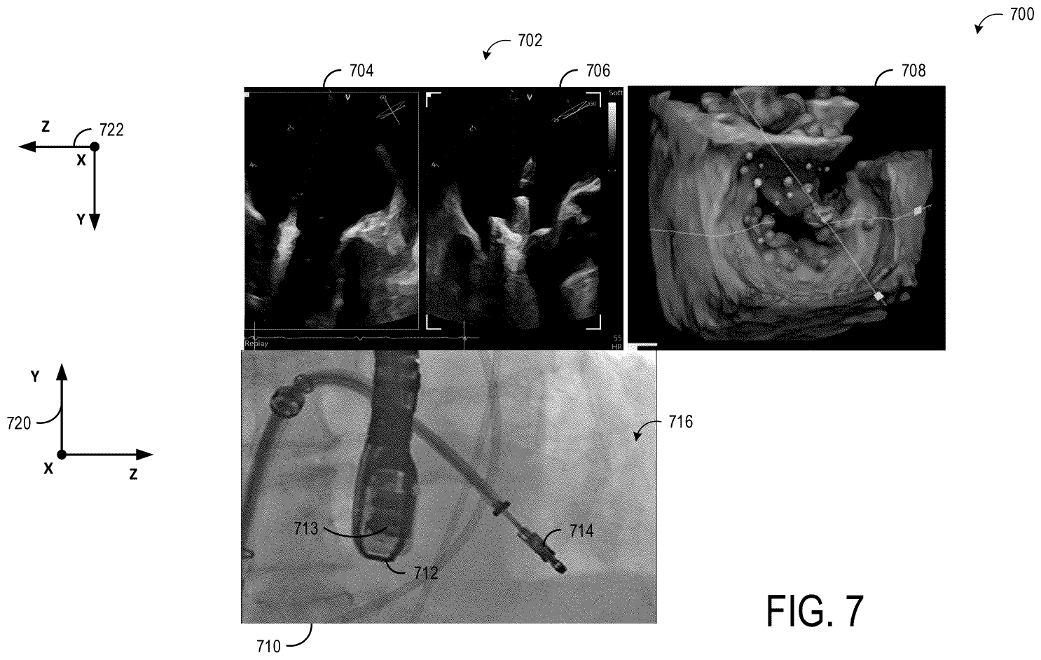

FIG. 4 shows an exemplary image processing system 402, in accordance with an embodiment. Image processing system 402 may be a non-limiting embodiment of image processing module 330 of FIG. 3. As described herein, image processing system 402 may be used to process one or more 2D images or 3D image volumes from imaging data acquired using one or more medical imaging systems 440. In particular, image processing system 402 may be used to reorient a 2D image generated in accordance with a first coordinate system to a second coordinate system. The second coordinate system may be a common coordinate system of the one or more 2D images or 3D image volumes, such as the common coordinate system 332 of FIG. 3 described above. Reorienting the 2D image may also include generating a desired 2D view of a 3D image volume by performing MPR, as described in greater detail below in reference to FIGS. 5 and 7. The image processing system 402 may be coupled to, or integrated into, an ultrasound imaging system such as imaging system 100, and/or a supplementary imaging system such as the fluoroscopy imaging system 151 of FIG. 1B.

In some embodiments, at least a portion of image processing system 402 is disposed at a device (e.g., workstation, edge device, server, etc.) communicably coupled to one or more medical imaging systems 440 via wired and/or wireless connections, and can receive or access image data generated by the one or more medical imaging systems 440. The one or more medical imaging systems 440 may include imaging system 100 of FIG. 1A. In one embodiment, the one or more medical imaging systems 440 include at least an ultrasound imaging system, a CT imaging system (e.g., CT imaging system 322), and a fluoroscopy imaging system (e.g., fluoroscopy imaging system 324). In other embodiments, a different type of imaging system may be additionally or alternatively included.

Image processing system 402 may be operably/communicatively coupled to a user input device 432 and a display device 434. In some examples, user input device 432 may be a shared input device of the one or more imaging systems 440. Similarly, display device 434 may be a shared display device of the one or more imaging systems 440.

Image processing system 402 includes a processor 404 configured to execute machine readable instructions stored in a non-transitory memory 406. Processor 404 may be single core or multi-core, and the programs executed thereon may be configured for parallel or distributed processing. In some embodiments, processor 404 may optionally include individual components that are distributed throughout two or more devices, which may be remotely located and/or configured for coordinated processing. In some embodiments, one or more aspects of processor 404 may be virtualized and executed by remotely-accessible networked computing devices configured in a cloud computing configuration.

Non-transitory memory 406 may store an AI module 408 and an MPR module 410. AI module 408 may include various AI models, such as machine learning (ML) and/or deep learning (DL) models. In various embodiments, the various AI models and/or disparity assessment models may include neural network models such as convolutional neural networks (CNNs), generative adversarial networks (GAN), transformers and traditional classifier; Bayesian networks, decision trees, and/or other statistical, probabilistic, or hierarchical models. AI module 408 may include trained and/or untrained neural networks and may further include various data, or metadata pertaining to the one or more AI models stored therein.

In one embodiment, an AI model of the various AI models may be used to define a desired plane of an image volume, to generate a desired view of an anatomy of a patient of a medical imaging system 440. For example, the AI model may automatically select one or more anatomical landmarks or points of an image volume of the patient anatomy that may be used to define the desired plane. The desired plane may be used by the MPR module 410 to generate an image including the desired view of the anatomy.

The MPR module 410 may be configured to perform multi-planar reformation on an image volume to generate a desired 2D view of the image volume, where the desired 2D view is defined by a second plane of the image volume that is preferred over a first plane of the image volume corresponding to a current 2D view.

User input device 432 may comprise one or more of a touchscreen, a keyboard, a mouse, a trackpad, or other device configured to enable a user to interact with image processing system 402. Display device 434 may include one or more display devices utilizing virtually any type of technology. In some embodiments, display device 434 may comprise a computer monitor. Display device 434 may be combined with processor 404, non-transitory memory 406, and/or user input device 432 in a shared enclosure, or may be peripheral display devices and may comprise a monitor, touchscreen, projector, or other display device known in the art, which may enable a user to view responses to queries submitted to image processing system 402, and/or interact with various data stored in non-transitory memory 406.

It should be understood that image processing system 402 shown in FIG. 4 is for illustration, not for limitation. Another appropriate image processing system may include more, fewer, or different components.

Referring now to FIG. 5, an exemplary simplified data flow diagram 500 depicts a flow of data within the imaging system 301 as images of a patient anatomy are generated for display during the image-guided transcatheter therapy task of FIG. 3. The imaging processing module 330 performs MPR to generate a 2D image 502 of an image volume 504 (e.g., such as the ultrasound image volume 307 or the CT image volume 326), where the 2D image 502 may correspond to a desired plane 505 defined by an operator 508.

The desired plane 505 may be defined according to three points. For example, the desired plane 505 may be defined based on three anatomical landmarks 506. Each anatomical landmark 506 may be a point in three dimensions within the image volume 504. The anatomical landmarks 506 may be selected by the operator 508 and/or with the aid of an ML model 510 (e.g., of the various AI models of AI module 408 of the image processing system 402 of FIG. 4).

For example, the image volume 504 may be the reoriented ultrasound image volume 346 of FIG. 3. The operator may guide the probe 302 through the body of the patient based on the reoriented images 340, 342, 344, and 346 as described above in reference to FIG. 3. The operator may reach a point in the patient's body where a 2D view of the reoriented ultrasound image volume 346 is not ideal. For example, the reoriented ultrasound image volume 346 may not show a desired portion of the target anatomy (e.g., target structure 102), or a delivery catheter, such as the delivery catheter 310. The operator may wish to display a different 2D view of the reoriented ultrasound image volume 346 that shows the desired portion of the target anatomy, or that shows the desired portion of the target anatomy from a different perspective. To generate the different 2D view, the operator typically defines the desired plane 505 that shows the desired portion of the target anatomy from a desired perspective. The operator may select a display of the reoriented ultrasound image volume 346 that includes standard (e.g., axial, sagittal, and coronal) planar views of reoriented ultrasound image volume 346. The operator may iteratively select one or more anatomical landmarks 506 by placing markers in one or more of the standard planar views, and/or rotate any of the standard planar views to generate an oblique view of the reoriented ultrasound image volume 346 that includes the desired plane 505. The operator may then display the different 2D view generated by the desired plane 505 on the display device. In some embodiments, an additional reorientation step may be performed to reorient the different 2D view to the common coordinate system 332. as described in reference to FIG. 3. The operator may continue to navigate the probe 302 and the delivery catheter 310 with the aid of the reoriented CT image 342.

However, manually performing the MPR to obtain the desired plane 505 may be cumbersome and time-consuming for the operator, and may interrupt a work flow of the operator. Additionally, the anatomical landmarks 506 may be defined imprecisely. To increase a precision in defining the desired plane 505, and to reduce a reliance on selecting anatomical landmarks and the manual, trial-and-error process described above, the catheter position data 317 acquired by sensor 316 may be advantageously used to specify a location of the tip of the delivery catheter 310 as one of the points used to define the desired plane 505 when performing the MPR. Additionally, because data acquired by the sensors includes orientation data, an orientation of the delivery catheter may be advantageously used in conjunction with the location of the tip of the delivery catheter 310 to define the desired plane 505.

Because the location and orientation of the sensor 316 is transmitted automatically to the image processing module 330 in the catheter position data 317, the desired plane 505 may be defined more quickly and with less input by operator 508 than if the sensor 316 was not included in imaging system 301. Further, in some embodiments, the ML model 510 may be used to select one or more anatomical landmarks 506 based on data including one or more of the probe position data 305, the device position data 315, and the catheter position data 317, further reducing a reliance on the input of operator 508.

Further, in other embodiments, the desired plane 505 may be defined by a combination of locations or orientations of any of the sensors 304, 314, and 316 of FIG. 3 and/or one or more anatomical landmarks 506. As one example, the desired plane 505 may be defined by the location of the transducer array (e.g., sensor 304), the location of the tip of the delivery catheter 310 (e.g., sensor 316), and an anatomical landmark 506 selected by the operator. As another example, the desired plane 505 may be defined by the location of the transducer array, the location of the tip of the delivery catheter 310, and an orientation of the delivery catheter. Performing the MLP using data from sensors such as sensors 304, 314, and 316 in the context of an interventional procedure is described in greater detail below, in reference to FIG. 6.

Turning now to FIG. 6, a method 600 is shown for reorienting one or more images on a display screen to be displayed in a common coordinate system during an image guided, interventional procedure, to reduce a cognitive load on an operator and/or imager performing the interventional procedure. The interventional procedure may be a transcatheter therapy task performed on a heart of a subject, as described above in reference to FIG. 3. The interventional imaging system may include, for example, an ultrasound imaging system such as imaging system 100 of FIG. 1A and a fluoroscopy imaging system such as fluoroscopy imaging system 151 of FIG. 1B. Additionally, one or more supplementary imaging systems, such as a CT imaging system, an MR imaging system, or a different type of imaging system may be used to generate images or image volumes that may be used to aid probe guidance during the interventional procedure.

In the present disclosure, embodiments of the method 600 may be described in a general context of computer executable instructions on a computing system or a processor. Generally, computer executable instructions may include routines, programs, objects, components, data structures, procedures, modules, functions, and the like that perform particular functions or implement particular abstract data types. Embodiments of the method 600 may also be practiced in a distributed computing environment where optimization functions are performed by remote processing devices that are linked through a wired and/or wireless communication network. In the distributed computing environment, the computer executable instructions may be located in both local and remote computer storage media, including memory storage devices. Method 600 may be performed by a processor of an image processing system, such as the processor 404 of the image processing system 400 of FIG. 4. Specifically, one or more steps of method 600 may be carried out in accordance with instructions stored in a non-transitory memory of the image processing system.

The order in which the method 600 is described is not intended to be construed as a limitation, and any number of the described steps may be combined in any order to implement the method 600 disclosed herein, or an equivalent alternative method. Additionally, certain blocks may be deleted from the method 600 or augmented by additional blocks with added functionality without departing from the spirit and scope of the subject matter described herein.

Method 600 begins at 602, where method 600 includes receiving one or more image volumes from pre-operative imaging studies performed on the patient prior to performing the interventional procedure. Performing the pre-operative imaging studies may include, for example, performing a CT scan using a CT imaging system (e.g., CT imaging system 322), and reconstructing a CT image volume of the ROI. In some embodiments, the CT image volume and/or 2D images of the CT image volume may be displayed on a display device (e.g., display device 196 of FIG. 1B) of the interventional imaging system during the procedure, to aid an operator in guiding the ultrasound probe or a delivery catheter (e.g., delivery catheter 310) of the interventional imaging system. Additionally or alternatively, a different imaging system, such as a MR imaging system, may be used to generate supplementary images used to aid in probe or catheter guidance.

At 604, method 600 includes receiving, upon an initiation of the interventional procedure, a fluoroscopy image of the ROI of the subject via the fluoroscopy imaging system. In some examples, additional images generated via the pre-operative imaging studies may also be received, which may also be used for image guidance.

At 606, method 600 includes receiving one or more ultrasound images from ultrasound image data received from an ultrasound probe, as the ultrasound probe is inserted into a body of the patient. For example, the ultrasound probe may be inserted into an esophagus or inferior vena cava of the patient, and navigated to the ROI (e.g., the heart). In some examples, the one or more ultrasound images include 2D images generated from a 2D ultrasound probe. In other examples, the one or more ultrasound images may include a 3D rendering of an ultrasound image volume generated from a 3D ultrasound probe, where a 2D view of the 3D ultrasound image volume may be displayed on the display device.

At 608, method 600 includes receiving probe, delivery catheter, and/or device location data from sensors positioned in the ultrasound probe, the delivery catheter, and/or a device used during the procedure. For example, the device may be a replacement valve, as described above in reference to FIG. 2. In other embodiments, the device may be a different device. Receiving the location data includes, at 610, receiving probe position data from a first sensor positioned in the ultrasound probe (e.g., sensor 230 of FIG. 2); at 612, receiving delivery catheter position data from a second sensor positioned in a tip of the delivery catheter (e.g., sensor 232); and at 614, receiving device position data from a third sensor positioned in the device (e.g., sensor 234).

At 616, method 600 includes generating one or more reoriented ultrasound, CT, and fluoroscopy images in a common coordinate system. Generating the reoriented ultrasound, CT, and fluoroscopy images (also referred to herein as the generated images) in the common coordinate system further comprises, at 618, automatically (e.g., without human intervention) selecting a common coordinate system of the various coordinate systems. In various embodiments, the common coordinate system may be a patient coordinate system defined by an orientation of the subject on a table of an imaging system used to image the subject, such as the fluoroscopy imaging system. Alternatively, the common coordinate system may be a coordinate system of the first, second, or third sensors. In other examples, the common coordinate system may be a pre-defined preferred coordinate system of the operator, based on the procedural task being performed.

In some examples, two or more of the generated images may already be in a same coordinate system, whereby the same coordinate system may be selected as the common coordinate system. Further, the selection of the common coordinate system may depend at least partially on an amount of computation used to reorient one or more of the images, where the common coordinate system may be selected to minimize a use of processing and memory resources of the image processing system. For example, it may be more computationally intensive to reorient a 3D image volume to a coordinate system of a sensor, than to reorient sensor data to a coordinate system of a 3D image volume. By performing the latter rather than the former, an amount of computational and memory resources of the image processing system (e.g., the processor 404 and the non-transitory memory 406 of FIG. 4) consumed may be reduced, making the image processing system more efficient.

At 620, generating the one or more reoriented ultrasound, CT, and fluoroscopy images in the common coordinate system further comprises performing an image transformation of one or more of the generated images to the common coordinate system. Performing the image transformation may be accomplished by determining, for each 2D view or image to be transformed, a homogenous transformation matrix that an underlying image volume may be multiplied by get a second, reoriented image volume.

In particular, geometrical constraints of the image volume, such as a centerline or lumen (e.g., lumen 225 of FIG. 2) of a blood vessel, artery or chamber of a heart, may be used to register a first coordinate system of a first image volume or sensor to a second coordinate system of a second image volume or sensor. For example, the vessel centerlines and/or lumen may be used to register a first coordinate system of an ultrasound image volume to a second coordinate system of a sensor, such as a sensor located in the tip of the delivery catheter. The ultrasound image volume may be reoriented to the second coordinate system of the sensor by calculating a distance from the sensor to a closest point on an inner surface of a patient anatomy of the ultrasound image volume, calculating the sum of all the measured distances, and determining a transformation matrix that minimizes the sum of the distances. After obtaining the transformation matrix, voxel data of the ultrasound image volume may be multiplied by the transformation matrix to obtain a reoriented ultrasound image volume, where the reoriented ultrasound image volume (e.g., reoriented ultrasound image volume 346 of FIG. 3) shares the second coordinate system of the sensor. This procedure may be performed on the ultrasound image volume, the CT image volume, or a different image volume.

Continuing with the example, since the position and orientation of the delivery catheter (based on the sensor) may be detected in the fluoroscopy image and the 2D ultrasound images, the fluoroscopy image and the 2D ultrasound images may be similarly reoriented to the second coordinate system of the sensor. For example, an orientation of the delivery catheter in the two dimensions of the 2D ultrasound images may be provided by the sensor, and the 2D ultrasound images may be rotated to match the common coordinate system of one or more other images or image volumes.

Thus, the inclusion of a sensor in a probe or an interventional tool (e.g., the delivery catheter, the ultrasound probe, an intravascular device, etc.) may establish a specific known location and orientation of the tool within each type of image, allowing potentially any of the images to be reoriented to a coordinate system of a different image. For example, the ultrasound image volume and the fluoroscopy image may be transformed to the second coordinate system of the sensor of the delivery catheter, or to a third coordinate system of the fluoroscopy image, or to a fourth coordinate system of the ultrasound probe. By showing the ultrasound image volume and the fluoroscopy image in a same coordinate system, anatomical directions (e.g., anterior-posterior, superior-inferior, and/or left-right) of the ultrasound image volume may be aligned (e.g., to a degree possible) with the fluoroscopy image. As a result, image guided manipulation of the delivery catheter may be more intuitive and natural, generating less cognitive load for the operator, which may result in shorter procedure times, reduced dosage, and increased availability of the interventional imaging system to patients. Without the aid of the sensor data, such a reorientation would have to be performed manually and/or via a much more computationally intensive and less precise procedure that relies on estimating the location and orientation of the tool from image data.

At 622, method 600 optionally includes replacing a first 2D view of an image volume (e.g., image volume 504 of FIG. 5) on the display device with a second 2D view desired by the operator or imager, using MPR. For example, the first 2D view of the ultrasound image volume may not show some or all of the delivery catheter, and the operator may wish to display the second, desired 2D view with a different orientation that shows the delivery catheter more fully, or more clearly. The desired view may also define a clinically useful spatial configuration of the anatomical structures in the image volume. The desired view including the anatomical structures in the clinically useful spatial configuration may also allow for accurate measurement of biometric parameters, and/or for an efficient assessment of a pathological condition of the subject.

At 624, replacing the first 2D view of the image volume with the desired 2D view includes receiving an operator input. The operator input may be received via one or more controls of the interventional imaging system (e.g., a button, a dial, etc.). In various embodiments, receiving the operator input may include receiving a selection of a pre-defined template of a plurality of pre-defined MPR templates, where the template may be used to automatically select the desired 2D view of the image volume. The template may be specified by the operator or imager via a user interface (UI) of the interventional imaging system. The template may be specified prior to the interventional procedure, or during the interventional procedure.

In various examples, the pre-defined templates may be selected via a menu of a software application used to view the image volume, such as a drop down menu. The operator input may select a menu item of the menu, and when the menu item is selected, MPR may be performed in accordance with the template corresponding to the menu item. Each template may include rules for defining a plane of the image volume that corresponds to the desired 2D view. In different templates, the plane may be defined based on a position and/or orientation of one or more probes, interventional tools, or devices (e.g., intravascular devices) used during the procedure, and/or a location of an anatomical landmark identified in the image volume.

Receiving the operator input may also include receiving one or more selected anatomical landmarks of the ROI. For example, the operator may place a marker on a selected anatomical landmark using a mouse or other input device of the interventional imaging system. In one embodiment, the operator may select an anatomical landmark by displaying standard sagittal, coronal, and axial views of the image volume, and manually adjusting planes in the sagittal, coronal, and axial views to define the anatomical landmark as a point of intersection of the planes. Alternatively, the operator may select an anatomical landmark in a different manner.

At 626, method 600 includes performing the MPR to obtain the desired 2D view based on the operator input. The desired 2D view may be based on a plane of the image volume that is defined by three points that are determined automatically by an AI module or MPR module of the image processing system (e.g., AI module 408 and MPR module 410 of FIG. 4, respectively). In other examples, one or more of the three points may be determined automatically by the imaging system, and other points may be selected by the operator as described above. Additionally, one or more of the three points may be determined automatically based on a location of the first, second, and/or third sensors positioned in the probe, catheter, and device, respectively. In other words, the anatomical landmarks may be included as “anchors”, for example, to ensure that the plane corresponding to the desired view intersects with target anatomical structures (e.g., mitral valve commissures, cooptation point of leaflets of the valve, etc.). Further, the plane could also be adjusted automatically in real-time to ensure a visibility of the tip of the delivery catheter to the operator.

In some embodiments, performing the MPR to obtain the desired 2D view based on the operator input may include receiving an additional set of position and orientation data from a fluoroscopic imaging system (e.g., fluoroscopy imaging system 151). The additional set of position and orientation data may be used, for example, to align the desired 2D view with an X-ray beam of the fluoroscopy imaging system, or to display a desired 2D view that is perpendicular to the X-ray beam.

Performing the MPR to obtain the desired 2D view based on the operator input may include defining a slice plane of the image volume based on at least one of the probe position data, the delivery catheter position data, and the additional set of position and orientation data from the fluoroscopic imaging system. For example, the slice plane may be defined based on the probe position data, where the slice plane is at a location of the probe and oriented in alignment with or perpendicular to an alignment of the probe. Alternatively, the slice plane may be defined based on a position of the probe, and an orientation of the X-ray beam of the fluoroscopy imaging system. The MPR may then be performed at the location of the slice plane. Alternatively, the slice plane may be used as a crop plane to generate a desired 3D rendered view of the image volume. In some embodiments, various desired 2D views may be generated concurrently.

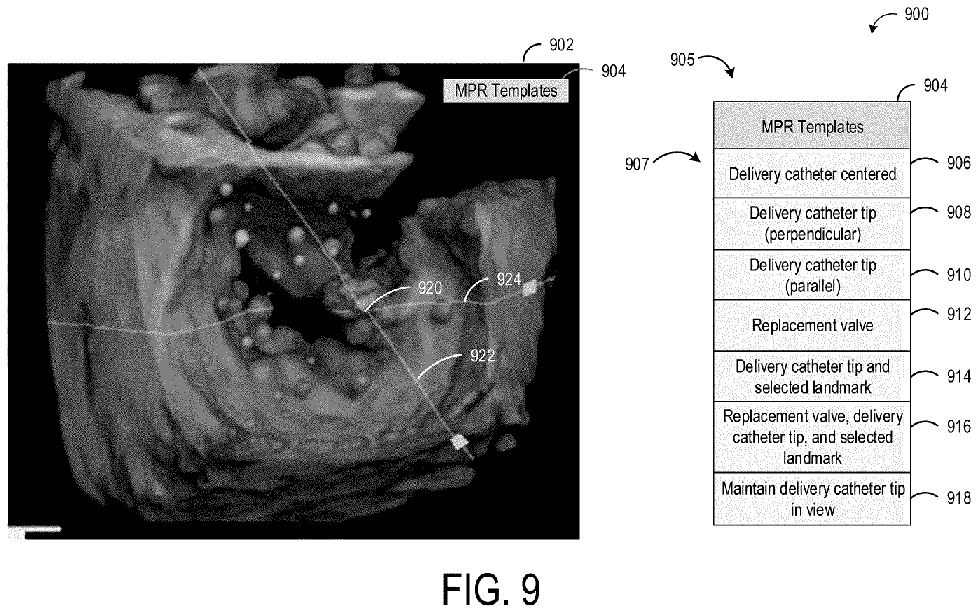

An example of how the desired 2D view may be generated based on the operator input is shown in FIG. 9. Referring briefly to FIG. 9, a GUI portion 900 of the interventional imaging system is shown, including an exemplary MPR template menu 904 displayed in a top right corner of a first 2D view of a 3D rendering of an ultrasound image volume 902. In other examples, the MPR template menu may be arranged in a different location with respect to the ultrasound image volume 902, or as part of a menu of a software application for displaying the ultrasound image volume 902. In one embodiment, the MPR template menu 904 may be a drop-down menu, that when selected with an input device (e.g., a mouse), displays various menu items 907 as shown in an expended view 905 of the MPR template menu 904.

In the depicted embodiment, six menu items are displayed, each menu item corresponding to a different template for an automated MPR procedure. In other embodiments, MPR template menu 904 may include a greater or lesser number of menu items. Each template may rely on position and orientation data obtained from sensors located in a probe, interventional tool, or device, as described above (e.g., the sensors 230, 232, and 234 of FIG. 2). The MPR template menu 904 may be used by an operator or imager during an interventional procedure, such as the transcatheter therapy task described above, to replace the first 2D view with a different, desired 2D view.

A first menu item 906 corresponds to a first template for performing MPR based on a position and orientation of a delivery catheter (e.g., the delivery catheter 224). The operator may select the first menu item 906, and MPR may be automatically performed to generate the desired 2D view of the image volume with the delivery catheter centered in the desired 2D view, meaning without relying on a manual intervention of the operator or imager. That is, a 2D view that shows the delivery catheter centered and/or with a preferred orientation may be automatically generated, by selecting a first plane of the image volume defined by the position and orientation of a sensor included in the delivery catheter (e.g., sensor 232). Additionally, in accordance with the first template, the MPR may be automatically performed when the delivery catheter disappears from the desired 2D view (or reaches a threshold distance from the center of the desired 2D view). For example, the operator may move the delivery catheter out of a view of the operator, and as a result, MPR may be performed to re-generate a new desired 2D view where the delivery catheter is centered. In this way, the delivery catheter (e.g., a tip of the delivery catheter) may be maintained in view of the operator, without manual intervention by the operator.

A second menu item 908 corresponds to a second template for performing MPR based on the position and orientation of the delivery catheter, where the operator may select the second menu item 908, and MPR may be performed to generate a 2D view of the ultrasound image volume based on a second plane that is perpendicular to a tip of the delivery catheter, based on the position and orientation of the sensor included in the delivery catheter. Similarly, a third menu item 910 corresponds to a third template for performing MPR based on the position and orientation of the delivery catheter, where the operator may select the third menu item 910, and MPR may be performed to generate a 2D view of the ultrasound image volume based on a third plane that is parallel to a tip of the delivery catheter, along an orientation of the delivery catheter, based on the position and orientation of the sensor included in the delivery catheter. A fourth menu item 912 corresponds to a fourth template for performing MPR based on a position of a replacement valve of a heart of the patient, where the operator may select the fourth menu item 912, and MPR may be performed to generate a 2D view of the ultrasound image volume with the valve replacement centered in the 2D view, based on position and orientation data of a sensor located in the replacement valve (e.g., sensor 234).

A fifth menu item 914 corresponds to a fifth template for performing MPR based on a position and orientation of the sensor included in the delivery catheter and a selected landmark 920. For example, the selected landmark 920 may be defined by the operator as the intersection of a first oblique plane 922, a second oblique plane 924, and a third oblique plane parallel to a view plane of the first 2D view shown in FIG. 9. The operator may select the fifth menu item 914, and MPR may be performed to generate a 2D view of the ultrasound image volume based on a plane defined by a line defined by the orientation of the delivery catheter, and the selected landmark 920, using the position and orientation data of a sensor located in the delivery catheter.

A sixth menu item 916 corresponds to a sixth template for performing MPR based on a first position and orientation of the sensor included in the delivery catheter, a second position and orientation of the sensor included in the replacement valve, and the selected landmark 920. The operator may select the sixth menu item 916, and MPR may be performed to generate a 2D view of the ultrasound image volume based on a plane defined by the three points (e.g., the two sensor positions and the anatomical landmark 920), using the position and orientation data of both of the sensor located in the delivery catheter and the sensor located in the replacement valve.

A seventh menu item 918 corresponds to a seventh template for performing MPR based on a first position and orientation of the sensor included in the ultrasound probe, and a second position and orientation of the sensor included in the delivery catheter. The operator may select the seventh menu item 918, and MPR may be automatically performed to generate the desired 2D view of the image volume with the delivery catheter centered in the desired 2D view.

It should be appreciated that the examples depicted in FIG. 9 are illustrative and not limiting, and in other examples, other templates for automated MPR procedures may be included as menu items of MPR template menu 904.

In this way, a user of the GUI portion 900 (e.g., the operator or imager) may efficiently access a plurality of different automated options for changing the 2D view of the image volume, based on a measured position and orientation of one or more sensors included in a probe or interventional tool used during the interventional imaging procedure and/or one or more anatomical landmarks selected by the user. In comparison to an alternative system not including the one or more sensors, adjusting the 2D view in such an automated fashion would entail a significantly more computationally intensive process of determining the position and orientation of interventional tools based on relative positions of anatomical landmarks with respect to the interventional tools, by estimating locations of points within the image volume and measuring the distances between the points. Further, MPR adjustments made on the basis of such measurements may be imprecise, due to factors including a difficulty of ultrasound imaging systems accurately rendering the interventional tools.

Returning to FIG. 6, at 628, method 600 includes inserting a computer aided design (CAD) model of one or more interventional tools in one or more image volumes, to more clearly and precisely depict the position and orientation of the interventional tool. For example, when the ultrasound probe is a 3D probe, and the 3D ultrasound image volume is updated in real time during the procedure, an interventional tool such as the delivery catheter may be visible in the ultrasound image volume. However, the delivery catheter may not be clearly depicted, as a result of materials of the delivery catheter (e.g., metal, plastic, etc.) not being imaged accurately by the ultrasound imaging system. As a result, the delivery catheter may be depicted with blurry or poorly defined boundaries and contours, or with distortions in a size or shape of the delivery catheter. Additionally, the delivery catheter may be depicted in the ultrasound image volume with a same color, shading, and texture as surrounding anatomical landmarks.

However, because the delivery catheter includes a sensor that provides accurate position and orientation data of the delivery catheter, a more accurate 3D image of the delivery catheter may be inserted into the ultrasound image volume at the position and orientation of the delivery catheter. In various examples, a pre-defined, realistic CAD model of the delivery catheter may be stored in a memory of the image processing system, and the pre-defined CAD model may be inserted into the ultrasound image volume such that the pre-defined CAD model may be viewed instead of the depiction of the delivery catheter in the ultrasound image volume. As a result, the operator may be guided in manipulating the delivery catheter by viewing a movement of the CAD model within the ultrasound image volume.

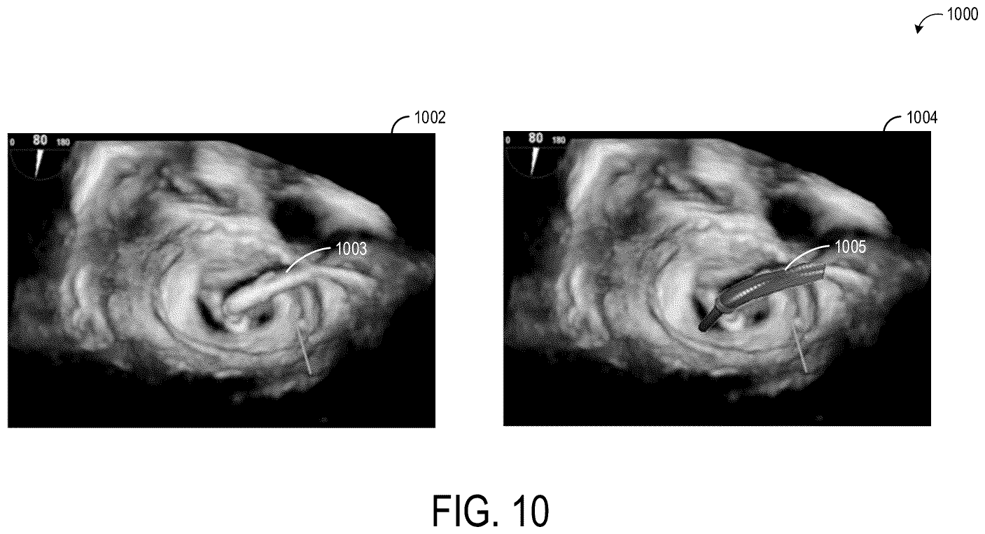

Referring briefly to FIG. 10, a CAD tool substitution example 1000 shows a first 3D rendering of an ultrasound image volume 1002, where a delivery catheter 1003 may be seen. However, the depiction of delivery catheter 1003 is not easily distinguishable from anatomical elements of the ultrasound image volume 1002. Additionally, boundaries of delivery catheter 1003 are not clearly delineated. As a result, an operator viewing ultrasound image volume 1002 may have difficulty determining how to manipulate the delivery catheter 1003 with respect to the anatomical elements. To address this problem, as shown in a second 3D rendering 1004 of the ultrasound image volume 1002, a CAD model 1005 of the delivery catheter 1003 may be inserted into the ultrasound image volume 1002 at the location of the delivery catheter 1003. Position and orientation data obtained from the sensor located in the delivery catheter may be advantageously used to adjust a size, position, and orientation of the CAD model 1005 within the ultrasound image volume 1002, such that the CAD model 1005 covers, encompasses, and replaces the depiction of delivery catheter 1003 in the ultrasound image volume 1002. Thus, when the operator manipulates the delivery catheter, a position and orientation of the CAD model 1005 may be accurately updated, to provide more accurate and precise image guidance to the operator.

Returning to FIG. 6, at 630, method 600 includes displaying the reoriented ultrasound, CT, and/or fluoroscopy image concurrently on a display device. For example, the 2D ultrasound images and/or 3D rendering of the ultrasound image volume, the fluoroscopy image, and the supplementary images may be displayed adjacent to each other on the display device, where one or more of the 2D ultrasound images and/or 3D rendering of the ultrasound image volume, the fluoroscopy image, and the supplementary images may be reoriented as described above. A position of each of the 2D ultrasound images and/or 3D image volume, the fluoroscopy images, and the supplementary images on the display device may be selected or adjusted by the operator, the imager, or a different participant in the interventional procedure. The ultrasound images and the fluoroscopy image may be updated on the display device as the interventional procedure is performed. In other words, as the operator adjusts a position of the ultrasound probe, the 2D ultrasound images displayed on the display device may change to reflect a current FOV of the ultrasound probe. In some examples, the probe may be placed in a desired position for viewing the ROI, and left in the desired position while a tool such as a delivery catheter is manipulated, whereby the selected 2D view may not change until the desired position is adjusted. The fluoroscopy image may be updated periodically or when requested by the operator or imager. Method 600 ends.

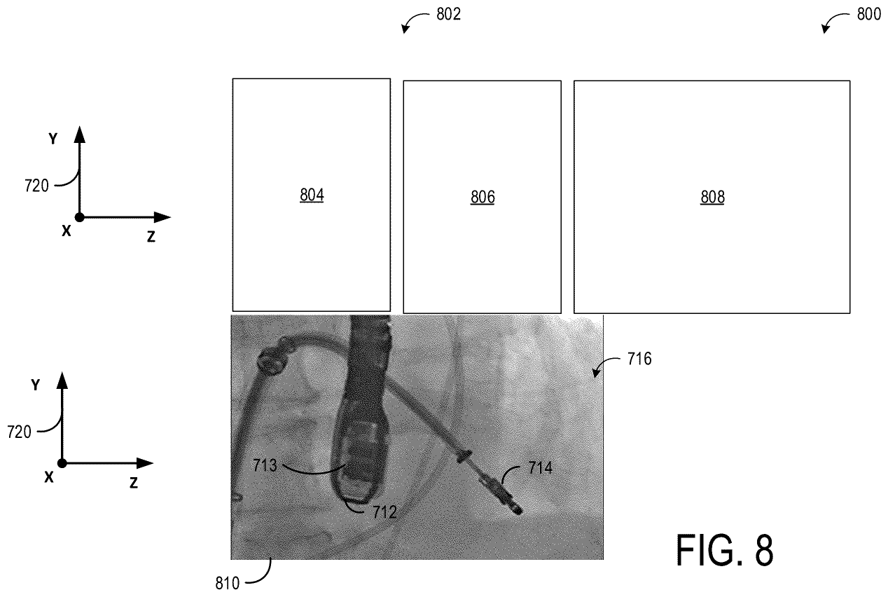

Referring now to FIGS. 7-8, exemplary images are shown that may be displayed to the operator and imager during the interventional procedure during various steps of method 600. FIG. 8 shows an effect of reorienting one or more images to a common coordinate system prior to display, in contrast with an alternative (e.g., prior art) conventional display of the images of FIG. 7.

FIG. 7 shows a first exemplary set of images 700 displayed on a display device of an interventional imaging system, such as the display device 196 of the interventional imaging system 150 of FIG. 1B, during a cardiac transcatheter therapy task such as the valve replacement task described above in reference to FIG. 2. The first set of exemplary images 700 includes a 2D ultrasound image 702 generated by an ultrasound imaging system of the interventional imaging system. The ultrasound system may be a non-limiting example of imaging system 100 of FIG. 1A. In FIG. 7, the 2D ultrasound image 702 may be a bi-plane ultrasound image, including a first ultrasound view 704 and a second ultrasound view 706. First ultrasound view 704 and second ultrasound view 706 may be standard orthogonal views generated via a transducer array 713 of an ultrasound probe 712 (e.g., catheter 106) as the ultrasound probe 712 is navigated or manipulated within a cavity 716 of a body of a patient of the cardiac transcatheter therapy task, such as an esophagus of the patient.

The first set of exemplary images 700 includes a 3D rendering 708 of an ultrasound image volume, which may be generated when the ultrasound probe 712 is a 3D probe. A 2D perspective view of the 3D rendering 708 of the ultrasound image volume may also be updated as the ultrasound probe 712 is navigated or manipulated. Additionally, the 2D perspective view may be adjusted by an operator of the ultrasound probe 712 (or an imager aiding the operator in the cardiac transcatheter therapy task) using MPR, as described in greater detail in reference to FIG. 9.

The first set of exemplary images 700 includes a fluoroscopy image 710, which may be generated by a fluoroscopy imaging system such as fluoroscopy imaging system 151 of FIG. 1B. The fluoroscopy image 710 is a 2D X-ray projection that may be updated periodically during the cardiac transcatheter therapy task, for example, when desired by the operator. The fluoroscopy image 710 shows the ultrasound probe 712, as well as a delivery catheter 714 inserted into the cavity 716 by the operator.

In FIG. 7, the fluoroscopy image 710 may be displayed according to a first coordinate system 720 of the fluoroscopy imaging system, which may be identical to the reference coordinate axes 199 of FIG. 1B (e.g., the patient coordinate system of the fluoroscopy imaging system 151). The first coordinate system 720 may be established by a physical configuration of the fluoroscopy imaging system, where axes of the first coordinate system may be aligned with a bed of the fluoroscopy imaging system. For example, an X axis of the first coordinate system 720 may be aligned along a length of the bed; a Y axis of the first coordinate system 720 may be aligned along a width of the bed; and a third axis of the first coordinate system 720 may be aligned orthogonally to a surface of the bed.

However, the 2D ultrasound image 702 and the 3D rendering 708 may be displayed according to a second coordinate system 722, which may be different from the first coordinate system 720. The second coordinate system 722 may be established by a physical configuration of the ultrasound probe 712, where axes of the second coordinate system may be aligned with the transducer array 713. For example, an X axis of the first coordinate system 720 may be aligned along a length of the transducer array 713; a Y axis of the first coordinate system 720 may be aligned along a width of the transducer array 713; and a third axis of the first coordinate system 720 may be aligned orthogonally to a surface of the transducer array 713. Additionally, the ultrasound image 702 may be displayed such that the direction towards the probe is pointing up.

It should be further appreciated that the first set of exemplary images 700 may include other images, such as, for example, a CT image volume, or an atlas, which may be displayed in accordance with different coordinate systems than the first coordinate system and the second coordinate system.

Because the fluoroscopy image 710 may be displayed according to a first coordinate system 720 and the 2D ultrasound image 702 and the ultrasound image volume 708 may be displayed according to the second coordinate system 722, a first movement of the delivery catheter 714 in a first direction by the operator in the real world may be depicted as a second movement of the delivery catheter 714 in a second direction in the fluoroscopy image 710, a third movement of the delivery catheter 714 in a third direction in the 2D ultrasound images 702, and a fourth movement of the delivery catheter 714 in a fourth direction in the 3D ultrasound image volume 708. The second, third, and fourth directions may be different, where the movement of the delivery catheter 714 in each of the generated images may not be aligned. In other words, in one of the generated images, the delivery catheter may appear to move laterally to the right; in another of the generated images, the delivery catheter may appear to move laterally to the left; and in yet another of the generated images, the delivery catheter may appear to move up, or down. As a result, a cognitive load on the operator as the operator manipulates the delivery catheter using image guidance may be increased, by having to mentally harmonize the different depictions into a mental conception of where the delivery catheter is within the heart of the patient. The increased cognitive load may increase an amount of time taken for the interventional procedure, increasing a cost of the procedure and reducing an availability of the interventional imaging system for other patients.

In contrast, FIG. 8 shows a second exemplary set of images 800 displayed on the display device of the interventional imaging system during the cardiac transcatheter therapy task of FIG. 7, where a portion of the set of images 800 have been oriented to a common coordinate system (e.g., the common coordinate system 332 of FIG. 3). The second exemplary set of images 800 includes a 2D bi-plane ultrasound image 802, including a first ultrasound view 804 and a second ultrasound view 806, and a 3D rendering 808 of an ultrasound image volume, all acquired via the ultrasound probe 712; and a fluoroscopy image 810.

In FIG. 8, the common coordinate system is the second coordinate system of the fluoroscopy image 810. In other words, fluoroscopy image 810 may be identical to fluoroscopy image 710 of FIG. 7 (e.g., both fluoroscopy image 710 and fluoroscopy image 810 have the same perspective view and fluoroscopy image 810 has not been reoriented). However, the 3D rendering 808 may be reoriented such that the 3D rendering 808 does not share the same perspective view as the 3D rendering 708. The 3D rendering 808 has been reoriented from the second coordinate system 722 to the first coordinate system 720 of the fluoroscopy image. Additionally, the first ultrasound view 804 and the second ultrasound view 806 may each be rotated, such that the two dimensions of the first ultrasound view 804 and the second ultrasound view 806 match the two dimensions of the fluoroscopy image 810.

As a result, because all of the generated images are displayed according to the first coordinate system 720 of the fluoroscopy image 810, the first movement of the delivery catheter 714 in a first direction by the operator in the real world may be depicted as moving in a same direction in each of the fluoroscopy image 810, the reoriented 2D ultrasound images 802 (e.g., the reoriented ultrasound image 340 of FIG. 3), and the reoriented 3D ultrasound image volume 808 (e.g., the reoriented ultrasound image volume 346 of FIG. 3), where the movement of the delivery catheter 714 in each of the generated images may be aligned. As a result, a cognitive load on the operator as the operator manipulates the delivery catheter may be decreased when guided by the generated images of FIG. 8, as opposed to being guided by the generated images of FIG. 7. That is, the operator may no longer have to mentally harmonize the different depictions into the mental conception of where the delivery catheter is within the heart of the patient. The decreased cognitive load may decrease an amount of time taken for the interventional procedure, thereby decreasing the cost of the procedure and increasing an availability of the interventional imaging system for other patients.

Thus, systems and methods are proposed to reorient one or more images generated by respective imaging systems and displayed on a display device to an operator during an interventional procedure, such that the images are shown with respect to a same coordinate system. Rather than relying on a computationally intensive registration process, sensors may be advantageously positioned in probes, interventional tools, or devices used in the procedure, such as an ultrasound probe and a delivery catheter. The sensors may be tracked to determine the precise 3D positions and orientation of the elements used in the procedure, from which coordinate systems of the different views and images may be harmonized, in accordance with methods described herein. Additionally, the sensors may be used to partially or totally automate performing MPR on an image volume of the one or more images. By including the sensors in the interventional tools and/or devices, a position and orientation of a tracked tool or device may be automatically determined, and one or more desired planes used by the MPR may be automatically configured based on the sensors. For example, MPR may be automatically performed to maintain a specified tool or device visible on the display device, as the operator moves the specified tool or device. In this way, the sensors may be advantageously used to automatically select a view plane that includes the catheter tip and/or target anatomies of the patient more rapidly and efficiently than may be accomplished using manual MPR. The operator may configure the MPR by selecting one of various predefined templates, rather than rotating views of the image volume until identifying a desired 2D view of the image volume. As a result, the images displayed on the display screen may provide more accurate image guidance for the procedure, with less effort by the operator, in a way that reduces a cognitive load on the operator. As a result of the more accurate image guidance and reduced cognitive load the procedure may be performed more rapidly and efficiently, reducing patient discomfort and increasing an availability of resources of the imaging systems for other patients. Additionally an amount of computational and memory resources of the image processing system consumed may be reduced by using the sensor data during image registration, making the image processing system more efficient.

The technical effect of automatically reorienting the images and performing MPR in an automated or semi-automated manner based on position and orientation data received from sensors positioned in a probe, interventional tool, and/or device used in an interventional procedure is that a cognitive load of an operator of the interventional procedure may be reduced, and an efficiency of the image guidance may be increased.

The disclosure also provides support for a method for performing multiplanar reformation (MPR) on an image volume generated via an interventional imaging system, the method comprising: during an image-guided interventional procedure performed by an operator of the interventional imaging system on a patient: receiving a first image volume from a probe of an ultrasound system of the interventional imaging system, receiving a first set of position and orientation data from a first sensor located in the probe, optionally, receiving a second set of position and orientation data from a second sensor located in a delivery catheter, optionally, receiving a third set of position and orientation data from a fluoroscopy imaging system, defining a slice plane of the first image volume based on at least one of the first set of position and orientation data, the second set of position and orientation data, and the third set of position and orientation data, and displaying a desired 2D view of the first image volume on a display device, the desired 2D view one of: a 3D rendered view of the first image volume, using the defined slice plane as a crop plane, an MPR of the first image volume at a location of the slice plane. In a first example of the method, defining the slice plane of the first image volume based on at least one of the first set of position and orientation data, the second set of position and orientation data, and the third set of position and orientation data further comprises defining the slice plane based on the second set of position and orientation data, wherein the slice plane has an orientation either aligned with or perpendicular to the orientation of the second sensor. In a second example of the method, optionally including the first example, defining the slice plane of the first image volume based on at least one of the first set of position and orientation data, the second set of position and orientation data, and the third set of position and orientation data further comprises defining the slice plane at a position based on the second set of position and orientation data, and at an orientation based on the third set of position and orientation data, the orientation either aligned with or perpendicular to an X-ray beam of the fluoroscopy imaging system. In a third example of the method, optionally including one or both of the first and second examples, a plurality of the desired 2D views are generated concurrently. In a fourth example of the method, optionally including one or more or each of the first through third examples, the method further comprises: receiving a second image volume acquired prior to the interventional procedure from a supplementary imaging system of the interventional imaging system, performing MPR on the second image volume based on the second set of position and orientation data to generate a second desired 2D view of the second image volume, and displaying the second desired 2D view of the second image volume on the display device, wherein the supplementary imaging system one of: a computed tomography (CT) system, a magnetic resonance imaging (MRI) system, and a second ultrasound imaging system. In a fifth example of the method, optionally including one or more or each of the first through fourth examples, the method further comprises: performing the MPR on the second image volume to generate the second desired 2D view based on the second set of position and orientation data, and not using the first set of position and orientation data. In a sixth example of the method, optionally including one or more or each of the first through fifth examples, the method further comprises: receiving a template selected by the operator that defines the second desired 2D view, and performing the MPR based on the second set of position and orientation data of the second sensor in accordance with the template. In a seventh example of the method, optionally including one or more or each of the first through sixth examples, the template is selected by the operator via a menu displayed on the display device. In a eighth example of the method, optionally including one or more or each of the first through seventh examples, the method further comprises: performing the MPR based on the first set of position and orientation data of the first sensor, the second set of position and orientation data of the second sensor, and an additional point in the second image volume. In a ninth example of the method, optionally including one or more or each of the first through eighth examples, the additional point in the second image volume is an anatomical landmark selected by the operator. In a tenth example of the method, optionally including one or more or each of the first through ninth examples, the additional point in the second image volume is set automatically by an image processing system of the interventional imaging system. In a eleventh example of the method, optionally including one or more or each of the first through tenth examples, the method further comprises: performing the MPR on a first plane of the second image volume, where the delivery catheter is centered in the first plane, based on the second set of position and orientation data of the second sensor, performing the MPR on a second plane of the second image volume that is perpendicular to a tip of the delivery catheter, based on the second set of position and orientation data of the second sensor, performing the MPR on a third plane of the second image volume that is parallel to the tip of the delivery catheter, along an orientation of the delivery catheter, based on the second set of position and orientation data of the second sensor, performing the MPR on a fourth plane of the second image volume in which an intravascular device placed within the patient using the delivery catheter is centered, based on a fourth set of position and orientation data of a third sensor included in the device, performing the MPR on a fifth plane of the second image volume, the fifth plane defined by the orientation of the delivery catheter and a position of a selected anatomical landmark, based on the second set of position and orientation data of the second sensor, and performing the MPR on a sixth plane of the second image volume, the sixth plane defined by the second set of position and orientation data of the second sensor, the fourth set of position and orientation data of the third sensor, and the selected anatomical landmark. In a twelfth example of the method, optionally including one or more or each of the first through eleventh examples, the method further comprises: performing the MPR in response to the delivery catheter not being visible in a displayed view of the second image volume, to maintain the delivery catheter within the displayed view. In a thirteenth example of the method, optionally including one or more or each of the first through twelfth examples, the method further comprises: inserting a computer-aided design (CAD) model of the delivery catheter into the second image volume, and adjusting a size, position, and orientation of the CAD model to cover and replace a depiction of the delivery catheter in the second image volume.

The disclosure also provides support for an interventional imaging system, comprising: an ultrasound probe including a first sensor that measures a first position and orientation of the first sensor, a delivery catheter including a second sensor that measures a second position and orientation of the second sensor, a supplementary imaging system, and an image processing system including a processor and a memory including instructions that when executed, cause the processor to: during a transcatheter therapy task performed on a patient by an operator of the interventional imaging system: receive a first image volume acquired via the ultrasound probe, perform multiplanar reformation (MPR) on a second image volume acquired prior to the transcatheter therapy task from the supplementary imaging system, based on at least one of the first position and orientation and the second position and orientation, to generate a desired 2D view of the second image volume, display the desired 2D view of the second image volume on a display device. In a first example of the system, the MPR is performed based on further instructions stored in the memory that when executed, cause the processor to: in response to the operator selecting a menu element displayed on the display device: display a menu of templates for performing the MPR, receive a selection of a template by the operator via the menu, and perform the MPR in accordance with the template. In a second example of the system, optionally including the first example, the MPR is performed in accordance with the template based on further instructions stored in the memory, that when executed, cause the processor to perform one of: MPR on a first plane of the second image volume, where the delivery catheter is centered in the first plane, based on the second position and orientation of the second sensor, MPR on a second plane of the second image volume that is perpendicular to a tip of the delivery catheter, based on the second position and orientation of the second sensor, MPR on a third plane of the second image volume that is parallel to the tip of the delivery catheter, along an orientation of the delivery catheter, based on the second position and orientation of the second sensor, MPR on a fourth plane of the second image volume in which an intravascular device placed within the patient using the delivery catheter is centered, based on a third position and orientation of a third sensor included in the device, MPR on a fifth plane of the second image volume, the fifth plane defined by the orientation of the delivery catheter and a position of an anatomical landmark selected by the operator, based on the second position and orientation of the second sensor, MPR on a sixth plane of the second image volume, the sixth plane defined by the second position and orientation of the second sensor, the third position and orientation of the third sensor, and the selected anatomical landmark, and MPR on a seventh plane of the second image volume, the seventh plane defined by the second position and orientation of the second sensor and the first position and orientation of the first sensor. In a third example of the system, optionally including one or both of the first and second examples, further instructions are stored in the memory, that when executed, cause the processor to insert a computer-aided design (CAD) model of the delivery catheter into the second image volume, and adjusting a size, position, and orientation of the CAD model to cover and replace a depiction of the delivery catheter in the second image volume.

The disclosure also provides support for a method, comprising: during an image-guided interventional procedure performed by an operator of an interventional imaging system on a patient: acquiring a first image volume from a probe of an ultrasound system of the interventional imaging system, receiving a second image volume acquired from the patient prior to the interventional procedure via a supplementary imaging system of the interventional imaging system, receiving position and orientation data of a sensor located in a delivery catheter used in the interventional procedure, a representation of the delivery catheter included in the second image volume, inserting a computer-aided design (CAD) model of the delivery catheter into the second image volume, a size, position, and orientation of the CAD model adjusted to cover and replace the representation of the delivery catheter in the second image volume, and displaying a view of the second image volume on a display device, the view including the CAD model. In a first example of the method, the supplementary imaging system one of: a computed tomography (CT) system, a magnetic resonance imaging (MRI) system, and a second ultrasound imaging system.

When introducing elements of various embodiments of the present disclosure, the articles “a,” “an,” and “the” are intended to mean that there are one or more of the elements. The terms “first,” “second,” and the like do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

In addition to any previously indicated modification, numerous other variations and alternative arrangements may be devised by those skilled in the art without departing from the spirit and scope of this description, and appended claims are intended to cover such modifications and arrangement. Thus, while the information has been described above with particularity and detail in connection with what is presently deemed to be the most practical and preferred aspects, it will be apparent to those of ordinary skill in the art that numerous modifications, including, but no limited to, form, function, manner of operation and use may be made without departing from the principles and concepts set forth herein. Also, as used herein, the examples and embodiments, in all respects, are meant to be illustrative and should not be construed to be limiting in any manner.

1. A method for performing multiplanar reformation (MPR) on an image volume generated via an interventional imaging system, the method comprising:
during an image-guided interventional procedure performed by an operator of the interventional imaging system on a patient:
receiving a first image volume from a probe of an ultrasound system of the interventional imaging system;
receiving a first set of position and orientation data from a first sensor located in the probe;
receiving a second set of position and orientation data from a second sensor located in a delivery catheter;
receiving a third set of position and orientation data from a fluoroscopy imaging system;
defining a slice plane of the first image volume based on at least one of the first set of position and orientation data, the second set of position and orientation data, and the third set of position and orientation data; and
displaying a desired 2D view of the first image volume on a display device, the desired 2D view one of:
a 3D rendered view of the first image volume, using the defined slice plane as a crop plane;
an MPR of the first image volume at a location of the slice plane.

2. The method of claim 1, wherein defining the slice plane of the first image volume based on at least one of the first set of position and orientation data, the second set of position and orientation data, and the third set of position and orientation data further comprises defining the slice plane based on the second set of position and orientation data, wherein the slice plane has an orientation either aligned with or perpendicular to the orientation of the second sensor.

3. The method of claim 1, wherein defining the slice plane of the first image volume based on at least one of the first set of position and orientation data, the second set of position and orientation data, and the third set of position and orientation data further comprises defining the slice plane at a position based on the second set of position and orientation data, and at an orientation based on the third set of position and orientation data, the orientation either aligned with or perpendicular to an X-ray beam of the fluoroscopy imaging system.

4. The method of claim 1, where a plurality of the desired 2D views are generated concurrently.

5. The method of claim 1, further comprising:
receiving a second image volume acquired prior to the interventional procedure from a supplementary imaging system of the interventional imaging system;
performing MPR on the second image volume based on the second set of position and orientation data to generate a second desired 2D view of the second image volume; and
displaying the second desired 2D view of the second image volume on the display device;
wherein the supplementary imaging system one of:
a computed tomography (CT) system;
a magnetic resonance imaging (MRI) system; and
a second ultrasound imaging system.

6. The method of claim 5, further comprising:
performing the MPR on the second image volume to generate the second desired 2D view based on the second set of position and orientation data, and not using the first set of position and orientation data.

7. The method of claim 6, further comprising receiving a template selected by the operator that defines the second desired 2D view, and performing the MPR based on the second set of position and orientation data of the second sensor in accordance with the template.

8. The method of claim 7, wherein the template is selected by the operator via a menu displayed on the display device.

9. The method of claim 5, further comprising performing the MPR based on the first set of position and orientation data of the first sensor, the second set of position and orientation data of the second sensor, and an additional point in the second image volume.

10. The method of claim 9, wherein the additional point in the second image volume is an anatomical landmark selected by the operator.

11. The method of claim 9, wherein the additional point in the second image volume is set automatically by an image processing system of the interventional imaging system.

12. The method of claim 5, further comprising:
performing the MPR on a first plane of the second image volume, where the delivery catheter is centered in the first plane, based on the second set of position and orientation data of the second sensor;
performing the MPR on a second plane of the second image volume that is perpendicular to a tip of the delivery catheter, based on the second set of position and orientation data of the second sensor;
performing the MPR on a third plane of the second image volume that is parallel to the tip of the delivery catheter, along an orientation of the delivery catheter, based on the second set of position and orientation data of the second sensor;
performing the MPR on a fourth plane of the second image volume in which an intravascular device placed within the patient using the delivery catheter is centered, based on a fourth set of position and orientation data of a third sensor included in the device;
performing the MPR on a fifth plane of the second image volume, the fifth plane defined by the orientation of the delivery catheter and a position of a selected anatomical landmark, based on the second set of position and orientation data of the second sensor; and
performing the MPR on a sixth plane of the second image volume, the sixth plane defined by the second set of position and orientation data of the second sensor, the fourth set of position and orientation data of the third sensor, and the selected anatomical landmark.

13. The method of claim 12, further comprising performing the MPR in response to the delivery catheter not being visible in a displayed view of the second image volume, to maintain the delivery catheter within the displayed view.

14. The method of claim 12, further comprising inserting a computer-aided design (CAD) model of the delivery catheter into the second image volume, and adjusting a size, position, and orientation of the CAD model to cover and replace a depiction of the delivery catheter in the second image volume.

15. An interventional imaging system, comprising:
an ultrasound probe including a first sensor that measures a first position and orientation of the first sensor;
a delivery catheter including a second sensor that measures a second position and orientation of the second sensor;
a supplementary imaging system; and
an image processing system including a processor and a memory including instructions that when executed, cause the processor to:
during a transcatheter therapy task performed on a patient by an operator of the interventional imaging system:
receive a first image volume acquired via the ultrasound probe;
perform multiplanar reformation (MPR) on a second image volume acquired prior to the transcatheter therapy task from the supplementary imaging system, based on at least one of the first position and orientation and the second position and orientation, to generate a desired 2D view of the second image volume;
display the desired 2D view of the second image volume on a display device.

16. The interventional imaging system of claim 15, wherein the MPR is performed based on further instructions stored in the memory that when executed, cause the processor to:
in response to the operator selecting a menu element displayed on the display device:
display a menu of templates for performing the MPR;
receive a selection of a template by the operator via the menu; and
perform the MPR in accordance with the template.

17. The interventional imaging system of claim 16, wherein the MPR is performed in accordance with the template based on further instructions stored in the memory, that when executed, cause the processor to perform one of:
MPR on a first plane of the second image volume, where the delivery catheter is centered in the first plane, based on the second position and orientation of the second sensor;
MPR on a second plane of the second image volume that is perpendicular to a tip of the delivery catheter, based on the second position and orientation of the second sensor;
MPR on a third plane of the second image volume that is parallel to the tip of the delivery catheter, along an orientation of the delivery catheter, based on the second position and orientation of the second sensor;
MPR on a fourth plane of the second image volume in which an intravascular device placed within the patient using the delivery catheter is centered, based on a third position and orientation of a third sensor included in the device;
MPR on a fifth plane of the second image volume, the fifth plane defined by the orientation of the delivery catheter and a position of an anatomical landmark selected by the operator, based on the second position and orientation of the second sensor;
MPR on a sixth plane of the second image volume, the sixth plane defined by the second position and orientation of the second sensor, the third position and orientation of the third sensor, and the selected anatomical landmark; and
MPR on a seventh plane of the second image volume, the seventh plane defined by the second position and orientation of the second sensor and the first position and orientation of the first sensor.

18. The interventional imaging system of claim 15, wherein further instructions are stored in the memory, that when executed, cause the processor to insert a computer-aided design (CAD) model of the delivery catheter into the second image volume, and adjusting a size, position, and orientation of the CAD model to cover and replace a depiction of the delivery catheter in the second image volume.

19. A method, comprising:
during an image-guided interventional procedure performed by an operator of an interventional imaging system on a patient:
acquiring a first image volume from a probe of an ultrasound system of the interventional imaging system;
receiving a second image volume acquired from the patient prior to the interventional procedure via a supplementary imaging system of the interventional imaging system;
receiving position and orientation data of a sensor located in a delivery catheter used in the interventional procedure, a representation of the delivery catheter included in the second image volume;
inserting a computer-aided design (CAD) model of the delivery catheter into the second image volume, a size, position, and orientation of the CAD model adjusted to cover and replace the representation of the delivery catheter in the second image volume; and
displaying a view of the second image volume on a display device, the view including the CAD model.

20. The method of claim 19, wherein the supplementary imaging system one of:
a computed tomography (CT) system;
a magnetic resonance imaging (MRI) system; and
a second ultrasound imaging system.
